(19) United States
(12) Patent Application Publication
(10) Pub. No.: US 2021/0121087 A1
(43) Pub. Date: Apr. 29, 2021

(54) SYSTEM AND METHODS FOR MODEL-BASED NONINVASIVE ESTIMATION AND TRACKING OF INTRACRANIAL PRESSURE

(71) Applicant: Massachusetts Institute of Technology, Cambridge, MA (US)

(72) Inventors: Thomas Heldt, Cambridge, MA (US); Syed Muhammad Imaduddin, Cambridge, MA (US); Andrea Fanelli, Oakland, CA (US)

(73) Assignee: Massachusetts Institute of Technology, Cambridge, MA (US)

(21) Appl. No.: 17/051,847

(22) PCT Filed: Apr. 30, 2019
(86) PCT No.: PCT/US19/29776

§ 371 Date: Oct. 30, 2020

Related U.S. Application Data
(60) Provisional application No. 62/665,996, filed on May 2, 2018.

(51) Int. Cl. A61B 5/03; A61B 5/021; A61B 5/0285; A61B 5/02; A61B 5/00

(52) U.S. Cl. CPC A61B 5/031; A61B 5/021; A61B 5/7275; A61B 5/02007; A61B 5/7278; A61B 5/0285

(57) ABSTRACT

Techniques for estimating intracranial pressure using arterial blood pressure and cerebral blood flow velocity measurements. The techniques may include obtaining a first set of data identifying arterial blood pressure and cerebral blood flow velocity of a patient during a first period of time and estimating an initial intracranial pressure value for the patient. The techniques further include obtaining a second set of data identifying arterial blood pressure and cerebral blood flow velocity of the patient during a second period of time, estimating an updated intracranial pressure value for the patient by determining a change in intracranial pressure of the patient based on the second set of data and the initial intracranial pressure value, and outputting information indicating the updated intracranial pressure value.

### RELATED APPLICATIONS

The present application claims the benefit under 35 U.S.C. § 119(e) to U.S. Application Ser. No. 62/665,996, filed May 2, 2018 under Attorney Docket No. M0437.70138US00 and titled “SYSTEM AND METHODS FOR MODEL-BASED NONINVASIVE ESTIMATION AND TRACKING OF INTRACRANIAL PRESSURE,” which is hereby incorporated herein by reference in its entirety.

### FIELD

Aspects of the technology described herein relate to techniques for estimating intracranial pressure (ICP) using data obtained through noninvasive or minimally invasive measurements of a patient.

### BACKGROUND

Intracranial pressure (ICP) is the hydrostatic pressure of cerebrospinal fluid (CSF), which is the fluid that surrounds and cushions the brain tissue of a human or animal. When the ICP becomes elevated in an individual, blood flow to the brain can become limited and lead to cerebral ischemic injury. Additionally, brain structures may become displaced (herniation) because of pressure differences within the cranial cavity and spinal canal, which may potentially lead to coma, cessation of breathing, and/or death. Elevation of ICP may occur in various neuropathological conditions, including hydrocephalus, traumatic brain injury, hemorrhagic stroke, and brain tumors. In managing these types of neuropathological conditions, it can be important to monitor the ICP of the individual to assess the cerebrovascular and cerebrospinal state of the individual and to determine if the ICP becomes elevated to a point that puts the individual at a high risk level.

Current clinical practices for monitoring ICP in a patient involve significantly invasive techniques which include penetrating the person's skull and inserting a catheter or pressure sensor to measure ICP directly in the cerebrospinal fluid space, such as the ventricles. Alternatively, pressure sensors can be placed into the brain tissue to measure brain tissue pressure as a surrogate for ICP. These techniques generally involve a physician with neurosurgical expertise to perform and have a risk of infection arising from entering a person's skull, which can limit these types of ICP measurements to individuals who are severely ill and are generally not performed across a broader group of patients where assessing their ICP may be beneficial. For example, monitoring ICP in a person who has chronic headaches may assist a physician in diagnosing or treating the person. However, it is unlikely that the person is in a medical state that would justify the medical resources involved in performing ICP measurements or the risk associated with obtaining such measurements.

Some less invasive techniques for estimating ICP involve using other physiological measurements that correlate with ICP or may otherwise act as a proxy for ICP. For example, one noninvasive method involves assessing the diameter of the optic nerve sheath. Another method involves applying external pressure on an individual's eyeball to balance retro-orbital pressure with ICP. In addition, there have been some computational techniques for estimating ICP that use physiological signals that can be obtained through noninvasive or less invasive means and apply these signals to a physiological model. However, these techniques have not been adapted in a clinical setting because they may lack the ability to obtain reliable estimates for ICP as well as the ability to perform continuous monitoring of an individual's ICP.

### SUMMARY

Some embodiments are directed to a system comprising: at least one hardware processor; and at least one non-transitory computer-readable storage medium storing processor-executable instructions that, when executed by the at least one hardware processor, cause the at least one hardware processor to perform a method. The method comprises obtaining a first set of data identifying arterial blood pressure and cerebral blood flow velocity of a patient during a first period of time; estimating an initial intracranial pressure value for the patient by using a statistical model to compute a posterior distribution of intracranial pressure values based on the first set of data and a prior distribution of intracranial pressure values; obtaining a second set of data identifying arterial blood pressure and cerebral blood flow velocity of the patient during a second period of time; estimating an updated intracranial pressure value for the patient by determining a change in intracranial pressure of the patient based on the second set of data and the initial intracranial pressure value; and outputting information indicating the updated intracranial pressure value.

Some embodiments are directed to at least one non-transitory computer-readable storage medium storing processor-executable instructions that, when executed by at least one hardware processor, cause the at least one hardware processor to perform: obtaining a first set of data identifying arterial blood pressure and cerebral blood flow velocity of a patient during a first period of time; estimating an initial intracranial pressure value for the patient by using a statistical model to compute a posterior distribution of intracranial pressure values based on the first set of data and a prior distribution of intracranial pressure values; obtaining a second set of data identifying arterial blood pressure and cerebral blood flow velocity of the patient during a second period of time; estimating an updated intracranial pressure value for the patient by determining a change in intracranial pressure of the patient based on the second set of data and the initial intracranial pressure value; and outputting information indicating the updated intracranial pressure value.

Some embodiments are directed to a method, comprising: obtaining a first set of data identifying arterial blood pressure and cerebral blood flow velocity of a patient during a first period of time; estimating an initial intracranial pressure value for the patient by using a statistical model to compute a posterior distribution of intracranial pressure values based on the first set of data and a prior distribution of intracranial pressure values; obtaining a second set of data identifying arterial blood pressure and cerebral blood flow velocity of the patient during a second period of time; estimating an updated intracranial pressure value for the patient by determining a change in intracranial pressure of the patient based on the second set of data and the initial intracranial pressure value; and outputting information indicating the updated intracranial pressure value.

Some embodiments are directed to a system comprising: at least one hardware processor; and at least one non-transitory computer-readable storage medium storing processor-executable instructions that, when executed by the at least one hardware processor, cause the at least one hardware processor to perform a method. The method comprises obtaining data that includes an arterial blood pressure waveform and a cerebral blood flow velocity waveform of a patient during a first period of time. The arterial blood pressure waveform and the cerebral blood flow velocity waveform are obtained at different locations of the patient. The method further comprises estimating an intracranial pressure value for the patient by using a statistical model to compute a posterior distribution of intracranial pressure values based on a likelihood of intracranial pressure given the data and a prior distribution of intracranial pressure values. Using the statistical model includes using at least one time offset value between the arterial blood pressure waveform and the cerebral blood flow velocity waveform. The method further comprises outputting information indicating the updated intracranial pressure value.

Some embodiments are directed to at least one non-transitory computer-readable storage medium storing processor-executable instructions that, when executed by at least one hardware processor, cause the at least one hardware processor to perform a method. The method comprises obtaining data that includes an arterial blood pressure waveform and a cerebral blood flow velocity waveform of a patient during a first period of time. The arterial blood pressure waveform and the cerebral blood flow velocity waveform are obtained at different locations of the patient. The method further comprises estimating an intracranial pressure value for the patient by using a statistical model to compute a posterior distribution of intracranial pressure values based on a likelihood of intracranial pressure given the data and a prior distribution of intracranial pressure values. Using the statistical model includes using at least one time offset value between the arterial blood pressure waveform and the cerebral blood flow velocity waveform. The method further comprises outputting information indicating the updated intracranial pressure value.

Some embodiments are directed to a method, comprising: obtaining data that includes an arterial blood pressure waveform and a cerebral blood flow velocity waveform of a patient during a first period of time. The arterial blood pressure waveform and the cerebral blood flow velocity waveform are obtained at different locations of the patient. The method further comprises estimating an intracranial pressure value for the patient by using a statistical model to compute a posterior distribution of intracranial pressure values based on a likelihood of intracranial pressure given the data and a prior distribution of intracranial pressure values. Using the statistical model includes using at least one time offset value between the arterial blood pressure waveform and the cerebral blood flow velocity waveform. The method further comprises outputting information indicating the updated intracranial pressure value.

### BRIEF DESCRIPTION OF DRAWINGS

Various aspects and embodiments will be described with reference to the following figures. The figures are not necessarily drawn to scale.

FIG. 1 is a diagram of an illustrative patient data processing pipeline for estimating intracranial pressure, in accordance with some embodiments of the technology described herein.

FIG. 2 is an exemplary plot of arterial blood pressure (ABP) versus time.

FIG. 3 is an exemplary plot of cerebral blood flow velocity (CBFV) versus time.

FIG. 4 is a diagram of an exemplary statistical model used in estimating intracranial pressure, in accordance with some of the embodiments of the technology described herein.

FIG. 5 is a diagram of an illustrative patient data processing pipeline for estimating time shifts and prediction errors for determining a likelihood of intracranial pressure, in accordance with some embodiments of the technology described herein.

FIG. 6 is an exemplary plot of arterial blood pressure (ABP), cerebral blood flow velocity (CBFV), and a range of time shifts between ABP and CBFV.

FIG. 7 is an exemplary diagram of an optimization routine used in estimating intracranial pressure, in accordance with some of the embodiments of the technology described herein.

FIG. 8 is a diagram of an illustrative data processing pipeline for estimating intracranial pressure using prediction errors, in accordance with some of the embodiments of the technology described herein.

FIG. 9 is an exemplary plot of prediction errors versus time offsets and intracranial pressure.

FIG. 10 is an exemplary plot of likelihood of intracranial pressure versus time offsets and intracranial pressure that corresponds to the prediction errors shown in FIG. 9.

FIG. 11 is an exemplary plot of likelihood of intracranial pressure versus intracranial pressure.

FIG. 12 is an exemplary plot of likelihood of intracranial pressure versus intracranial pressure.

FIG. 13 is an exemplary plot of a prior distribution of intracranial pressure versus intracranial pressure, in accordance with some of the embodiments of the technology described herein.

FIG. 14 is an exemplary plot of a prior distribution of intracranial pressure versus intracranial pressure, in accordance with some of the embodiments of the technology described herein.

FIG. 15 is an exemplary plot of a posterior distribution of intracranial pressure versus intracranial pressure.

FIG. 16 is a flow chart of an illustrative process for estimating intracranial pressure, in accordance with some embodiments of the technology described herein.

FIG. 17 is a flow chart of an illustrative process for estimating intracranial pressure, in accordance with some embodiments of the technology described herein.

FIG. 18 is a flow chart of an illustrative process for evaluating noise in patient data, in accordance with some embodiments of the technology described herein.

FIG. 19 is a block diagram of an illustrative computer system that may be used in implementing some embodiments of the technology described herein.

FIG. 20 is a diagram illustrating a discrete-time model of the cerebral vasculature. Samples of cAMP, pₐ, and the CBFV, q, are related by a time-varying FIR filter, whose coefficients, αₘ and βₙ are assumed to remain constant during individual estimation windows. The mean ICP, I[m], is also assumed to be constant during an estimation window, and its evolution is modeled by an AR process.

FIG. 21 is a plot of prior distribution used for baseline estimation. Negative ICP values, as well as values exceeding 30 mmHg, have been assigned probability larger than that found in our data, in order to make our method broadly applicable. The distribution is composed of a mixture of two Gaussian distributions that model low and high ICP values, respectively.

FIG. 22 is a diagram illustrating an overview of model validation scheme. CBFV and rABP were collected and passed through a signal conditioning stage. The resulting data were passed to the estimation method, and the nICP estimates were then compared with reference mean ICP values. The reference mean values were computed by averaging the corresponding invasively measured ICP waveform.

FIGS. 23A, 23B, and 23C are exemplary plots of nICP estimates versus time. Invasive reference ICP measurements are shown in gray. Mean reference ICP are shown by the squares and nICP values are shown by the circles.

FIGS. 24A and 24B are exemplary plots of Bland-Altman analysis of estimation performance on per-estimation-window and per-recording-window bases, respectively.

FIG. 25 is a plot of estimation performance across all three patients. Bars indicate the estimation bias, and unit standard deviation extents are shown by the error bars.

FIG. 26 is a plot illustrating fraction of nICP estimates below a specified RMSE in per-estimation-window (solid), per-record (dotted), and per-patient (dashed) bases.

### DETAILED DESCRIPTION

Computational techniques that incorporate physiological signals, such as arterial blood pressure (ABP) and cerebral blood flow velocity (CBFV), may be used in estimating intracranial pressure (ICP) for an individual. However, the inventors have recognized that conventional computational techniques for estimating ICP have limitations in the accuracy of the estimated ICP value and the ability to tailor the ICP estimates to a particular patient.

For example, some conventional techniques for estimating ICP involve mapping measurements of ABP and CBFV to measurements of ICP for a group of patients, the mapping then may be used in determining an ICP estimate for a different patient by applying the mapping to ABP and CBFV measurements of the patient. However, the inventors have recognized that to determine the mapping involves obtaining ICP measurements from patients by invasively penetrating the patient's skull and that generally these patients are being hospitalized in an environment, such as in the intensive care unit, where their own ICP is being monitored and controlled to obtain a stable ICP value. As a result, these ICP measurements used in developing the mapping do not necessarily accurately represent the range of ICP measurements in the overall population of people, or even in those patients with acute injury or exacerbation of underlying conditions, which can lead to inaccuracies in estimating ICPs for other individuals. In some instances, individuals that have ICP, ABP, and/or CBFV not represented in the patient data used in developing the mapping may have inaccurate estimates for ICP when their ABP and CBFV data is applied to the mapping because the mapping does not specifically account for their own particular physiology.

In addition, some conventional techniques for estimating ICP that rely on using ABP and CBFV in computing an ICP estimate do not account for any misalignment in the ABP and CBFV waveforms as this data is obtained in real-time from different devices. In particular, the ABP and CBFV waveforms acquired from different devices may not represent cardiovascular physiology in an accurate manner. For example, ABP measurements can be obtained at an extremity of a person, such as the person's finger or wrist, while CBFV measurements can be obtained at the person's head, such as by using transcranial Doppler ultrasonography. A time shift between ABP measurements obtained at a location of the person that differs from where the CBFV measurements are obtained may create a physiologically induced time delay between the ABP waveform arriving at the cerebral artery and the ABP waveform at the actual measurement location. Although the ABP and CBFV measurements are obtained at the same time, this physiological time delay is represented in these measurements and can lead to misalignment between the ABP and CBFV waveforms in a manner that represents inaccurate or impossible physiology. In particular, cardiac cycles have quasi-regular, repeated characteristics in ABP and CBFV waveforms which are representative of the underlying cardiovascular physiology. During a cardiac cycle, a systolic peak in the CBFV waveform generally leads the corresponding systolic peak in the ABP waveform and the diastolic points in the ABP and CBFV waveforms are aligned with each other. A misalignment that arises from obtaining ABP and CBFV measurements from different locations of a person's body may create a combination of ABP and CBFV waveforms that represents physiologically inaccurate or impossible cardiac cycles. For example, one type of misalignment may include a systolic peak in the CBFV waveform following the corresponding systolic peak in the ABP waveform during the same cardiac cycle. Another type of misalignment may include the diastolic points in the ABP and CBFV waveforms not in alignment. These misalignments can lead to inaccurate estimates for ICP if not accounted for when computing an ICP using the misaligned ABP and CBFV waveforms.

Accordingly, the inventors have developed new computational techniques for estimating ICP, which accounts for the lack of physiological data representative of a cross-section of the population as well as possible misalignments in the physiological data (e.g., ABP, CBFV) being used in estimating ICP. These new computational techniques involve using a statistical model, which incorporates elements representative of cerebrovascular and cerebrospinal physiology, to estimate intracranial pressure values based on ABP and CBFV data from a patient. Estimating an intracranial pressure value may involve using the statistical model to compute an initial ICP for a patient and changes in ICP relative to that initial ICP value, which when added to the initial ICP value may provide an estimate of ICP for the patient at a particular time. For example, an initial ICP value may be obtained for ABP and CBFV data associated with a first time period and then subsequent ABP and CBFV data obtained from the patient may be used to track changes relative to that initial ICP value for subsequent time periods. Those changes in ICP may be combined with the initial ICP value to estimate an ICP value at for a particular time period. In this manner, a patient's ICP may be monitoring in real-time and dynamically updated using noninvasive physiological measurements.

The computational techniques developed by the inventors involve computing the initial ICP value using ABP and CBFV data from a patient, and may include incorporating data obtained from other people, and computing the changes in ICP value using additional ABP and CBFV data from the patient with or without other data from another person. The inventors have recognized and appreciated that while data from someone other than the patient whose ICP is being monitored may be important in computing an initial ICP value, using such data may introduce biases in the ICP estimates and provide inaccurate ICP estimates. Estimating changes in ICP using the patient's own data may account and compensate for such biases as additional patient data is obtained over time because there is less of reliance on data from people other than the patient.

The inventors have further recognized and appreciated that inaccuracy in estimating ICP can arise from misalignment in ABP and CBFV waveforms. For example, obtaining ABP and CBFV measurements at different locations of a person at the same time may introduce a physiological time delay that, when not accounted for, can lead to inaccurate estimates in ICP. As another example, the devices used in obtaining the ABP and CBFV measurements may have internal time delays. Accordingly, some embodiments of the technology described herein relates to introducing time offsets as parameters of the statistical model to account for misalignment in ABP and CBFV waveforms. In particular, computing an estimate for ICP in a patient may involve determining one or more time offset values that align the ABP and CBFV waveforms in time to meet certain physiological constraints.

Some embodiments described herein address all of the above-described issues that the inventors have recognized with estimating ICP. However, not every embodiment described herein addresses every one of these issues, and some embodiments may not address any of them. As such, it should be appreciated that embodiments of the technology described herein are not limited to addressing all or any of the above-discussed issues with estimating ICP.

Some embodiments involve obtaining data identifying arterial blood pressure (ABP) and cerebral blood flow velocity (CBFV) of a patient, estimating an initial intracranial pressure (ICP) value for the patient, estimating an updated ICP value for the patient by determining a change in ICP of the patient based on the data, and outputting information indicating the updated ICP value. The ABP and CBFV data may be obtained over multiple cardiac cycles, and estimating the initial ICP and changes in ICP may involve using data associated with one or more cardiac cycles.

Estimating the initial ICP value may involve using a statistical model to compute a posterior distribution of ICP values based on a set of ABP and CBFV data and a prior distribution of ICP values. In some embodiments, the prior distribution of ICP values correspond to data obtained from at least one person other than the patient. The statistical model relates arterial blood pressure and cerebral blood flow velocity to intracranial pressure. In some embodiments, the statistical model includes a parameter representing cerebrovascular resistance, a parameter representing cerebrovascular compliance, and a parameter representing intracranial pressure. Estimating an updated ICP value for the patient may involve determining a change in ICP of the patient based on a different set of ABP and CBFV data and the initial ICP value. These techniques may be applied to monitoring a patient's ICP in real-time such that the patient's ICP value is updated to reflect current ABP and CBFV data obtained from the patient over time. For example, the initial ICP value may be computed from a patient's ABP and CBFV data obtained during a first time period and individual updates in ICP may be computed from the patient's ABP and CBFV data obtained from subsequent time periods, where an update in ICP is computed for the individual subsequent time periods and subsequently combined with the initial ICP value to estimate an ICP value for a particular time period. Accordingly, some embodiments involve estimating a series of ICP values for the patient by determining changes in ICP of the patient based on patient data and combining changes in ICP with the initial ICP value. In some embodiments, estimating the series of ICP values involves dynamically updating an ICP value as patient data is obtained. The dynamic updating of the ICP value may be performed in an adaptive manner.

Some embodiments involve using Bayesian statistics in estimating ICP from patient data. In some embodiments, estimating an ICP value involves using the statistical model to compute a posterior distribution of ICP values based on a likelihood of ICP given the patient data and a prior distribution of ICP values. In some embodiments, the prior distribution of ICP values may be associated with data from other patients. Such a prior distribution may be used in determining the initial ICP value. In some embodiments, the prior distribution of ICP values may be generated from user input. For example, a uniform prior distribution having the same probability across all ICP values may be inputted by a user and used in determining a change in ICP.

Some embodiments may involve evaluating whether a time period in the patient data is of low quality, noisy, or otherwise may contribute to an inaccurate ICP estimate. For example, an ICP value may be estimated for a particular timeframe and time periods within that timeframe may each provide an estimated ICP value that may be combined to determine the estimated ICP value for the timeframe. In some embodiments, some of the time periods have data of low quality (e.g., where the patient moved suddenly and disrupted one or both of the ABP and CBFV measurements during the time period). The inventors have recognized and appreciated that it is important to remove or reduce these low quality time periods in estimating an ICP value for the entire timeframe. In some embodiments, estimating an ICP value for a timeframe may involve computing an ICP value using the patient data for a time period within the timeframe, determining a metric indicative of the level of noise in the patient data for the time period, and selecting to include the ICP value for the time period in estimating the ICP value for the timeframe based on comparing the metric to a threshold value. For example, if the metric is above a threshold value, then the ICP value for the time period is not included in estimating the ICP value for the timeframe.

Some embodiments involve determining changes in ICP using the statistical model and ABP and CBFV data to estimate values for parameters of the statistical model. The inventors have further recognized and appreciated that computational costs and overall efficiency in estimating ICP values may be reduced by using optimization techniques that allow for estimating values for parameters of the statistical model by evaluating different values for parameters independently. In some instances, monitoring ICP in a patient may involve computing ICP estimates during different time periods where the time periods have relevant time scales in the range of 5 seconds to 60 seconds. Estimating an ICP value for a particular time period may involve computing values for parameters of the statistical model to use in computing an ICP value or change in ICP during that time period. For different time periods, the values for the parameters of the statistical model may need to be updated to reflect the data associated with that time period. Accordingly, to allow for monitoring of a patient's ICP as data is obtained involves computing values for these parameters at least within the time scales where providing ICP estimates in real-time is desired. Determining an estimated ICP value for a certain time period may involve evaluating particular values for parameters using patient data during that time period. By providing particular values of parameters to evaluate, computational costs may be reduced, which may allow for ICP estimates to be obtained within a desired timeframe at the time scale of one or more cardiac cycles. In some embodiments, evaluating the parameter values for a particular time period may involve evaluating different combinations of possible parameter values for that time period using parallel computing techniques, which may improve computational efficiency and reduce computational time associated with providing an updated ICP value.

Some embodiments may involve predicting an ICP value using ABP and CBFV data from a patient. The predicted ICP value may be obtained by estimating a change in ICP for a future time using the patient data and the statistical model and combining the estimated change in ICP with one or more previously determined ICP values. In particular, values for parameters of the statistical model may be estimated using the patient data and those estimated parameter values may be used in estimating the change in ICP for the future time. These predictions in ICP may assist in monitoring ICP of a patient by assessing how the patient's ICP may change in the future, such as whether ICP is likely to remain at a stable value or become elevated. In some embodiments, estimating an updated ICP value may involve computing an data-derived ICP value for a time interval using the data obtained during that interval and a predicted change in ICP for the time interval using data from a prior time interval, determining an estimated change in ICP based on the predicted change in ICP and the data-derived ICP, and using this estimated change in ICP to estimate the updated ICP value.

In some embodiments, evaluating possible values for parameters of the statistical model may involve predicting a change in ICP for a time period using patient data from a prior time period. The predicted change in ICP may be compared to a data-derived change in ICP value estimated for the time period using data from that time period. If the predicted and data-derived changes in ICP estimates are similar, then the parameter values used in obtaining the predicted and data-derived changes in ICP estimates may be determined to have a high level of accuracy in estimating ICP. While, if the predicted and data-derived changes in ICP have significant variability, then the parameter values may be determined as having a low level of accuracy in estimating ICP.

In some embodiment, a time offset between ABP and CBFV waveforms is a parameter of the statistical model and evaluating parameter values may involve evaluating intracranial pressure values at different time offsets. The time offsets may be selected from a range of time offsets obtained by aligning the ABP and CBFV waveforms using physiological constraints. In some embodiments, a range of time offsets may be identified from aligning the ABP and CBFV waveforms and one or more time offset values may be selected from the range based on whether the ABP and CBFV waveforms meet a set of physiological constraints when a particular time offset is used in shifting the ABP and CBFV waveforms relative to one another. One type of physiological constraint that may be used in aligning the ABP and CBFV waveforms is having a systolic peak in CBFV occur prior to a systolic peak in ABP for the corresponding cardiac cycle. Another type of physiological constraint that may be used in aligning the ABP and CBFV waveforms is having a diastolic point in CBFV occur at substantially the same time as a diastolic point in ABP in the same cardiac cycle.

Some embodiments involve predicting physiological signals (e.g., ABP, CBFV) for a patient using the statistical model and patient data to evaluate multiple ICP values corresponding to different time offsets between ABP and CBFV waveforms. Prediction errors may be determined by comparing predicted values for the physiological signals to the patient data. The prediction errors may be used in computing a likelihood of ICP, which may be used in estimating an ICP. In some embodiments, computing the likelihood of ICP may involve determining a likelihood distribution of ICP for different time offsets from prediction errors associated with using the time offset in computing a physiological signal. In some embodiments, computing the likelihood of ICP involves combining the likelihood of ICP distribution for the different time offsets to determine the likelihood of ICP.

It should be appreciated that the various aspects and embodiments described herein be used individually, all together, or in any combination of two or more, as the technology described herein is not limited in this respect.

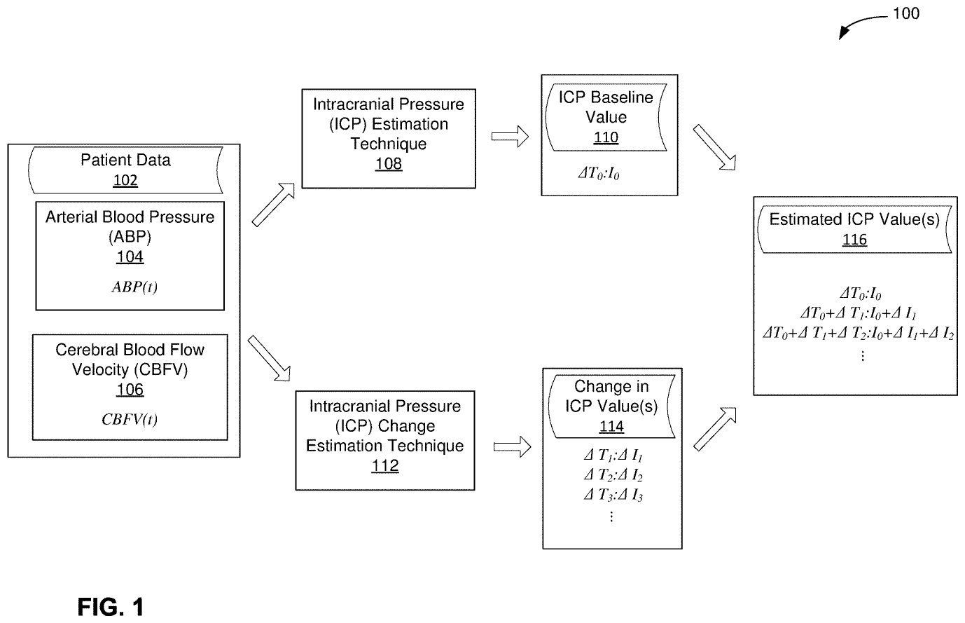

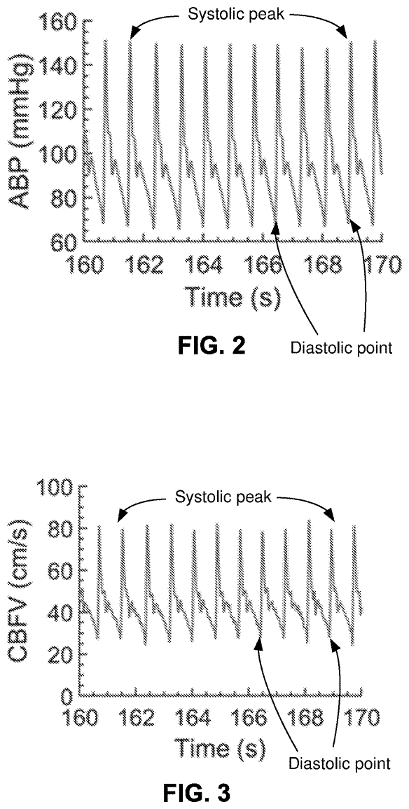

FIG. 1 is a diagram of an illustrative processing pipeline 100 for estimating intracranial pressure for a patient, which may include obtaining patient data and using a statistical model to compute estimated intracranial pressure values and changes in intracranial pressure values based on the patient data, in accordance with some embodiments of the technology described herein. As shown in FIG. 1, patient data 102 may be obtained and analyzed using pipeline 100. Patient data 102 may include arterial blood pressure (ABP) 104 and cerebral blood flow velocity (CBFV) 106. ABP data 104 for a patient can be obtained by measuring ABP at an extremity of a person, such as the person's finger or wrist. CBFV data for a patient can be obtained by using transcranial Doppler ultrasonography to measure CBFV at a person's head. Patient data 102 can be obtained over multiple cardiac cycles. A cardiac cycle, which is also referred to as a heartbeat, has both a diastole phase, which is when the heart relaxes and fills with blood, and a systole phase, which is when the heart contracts and pumps blood. FIG. 2 is an exemplary plot of ABP versus time illustrating a representative ABP waveform for multiple cardiac cycles. FIG. 3 is an exemplary plot of CBFV versus time illustrating a representative CBFV waveform for multiple cardiac cycles. Systolic peaks corresponding to the end of the systole phase and diastolic points corresponding to the end of the diastole phase are labeled in FIGS. 2 and 3.

Intracranial pressure (ICP) estimation technique 108 may be used to estimate ICP baseline value 110 using patient data 102. ICP estimation technique 108 may include using a statistical model and patient data 102 to compute ICP baseline value 110. Intracranial pressure (ICP) change tracking technique 110 may be used to estimate change in ICP value(s) 114 using patient data 102. ICP change tracking technique 110 may include using the statistical model and patient data 102 to compute changes in ICP value(s) 114. ICP baseline value 110 and change in ICP value(s) 114 may be used in determining estimated ICP value(s) 116, which are outputted by processing pipeline 100.

Some embodiments may involve using pipeline 100 for estimating a series of intracranial pressure values for a patient, which may allow for real-time monitoring of the patient's ICP. In some embodiments, estimating the series of ICP values involves dynamically updating an ICP value as patient data 102 is obtained. As shown in FIG. 1, ICP estimation technique 108 may estimate an initial ICP value, I₀, as ICP baseline value 110 corresponding time period, ΔT₀. In some embodiments, patient data 102 corresponding to multiple cardiac cycles may be used in estimating the ICP baseline value 110. For example, some implementations of ICP estimation technique 108 involves using a time period corresponding a number of cardiac cycles in the range of 10 to 100 cardiac cycles, or any value or range of values in that range. In some embodiments, the time period of patient data 102 used by ICP estimation technique 108 to estimate ICP baseline value 110 is approximately 20 cardiac cycles. ICP change tracking technique 112 may estimate changes in ICP value(s) 114 using patient data 102 for times periods that occur after the time period ΔT₀ used in estimating ICP baseline value 110. As shown in FIG. 1 ICP change tracking technique 112 may estimate change in ICP values, ΔI₁, ΔI₂, ΔI₃, . . . , corresponding to time periods ΔT₁, ΔT₂, ΔT₃, . . . which occur after ΔT₀. Estimated ICP value(s) 116 may be determined by combining ICP baseline value 110 and change in ICP value(s) 114. Some embodiments may involve serially combining, for each additional time period, the change in ICP value corresponding to the time period with the ICP baseline value 110 to arrive at an estimated ICP value for that time period. As shown in FIG. 1, estimated ICP value(s) 116 include I₀, I₀+ΔI₁, I₀+ΔI₁+ΔI₂, . . . for time periods ΔT₀, ΔT₁, ΔT₂, . . . , respectively. Additional discussion for estimating a baseline ICP value and tracking changes in ICP is described herein including in Sections A.4.4 and A.4.5.

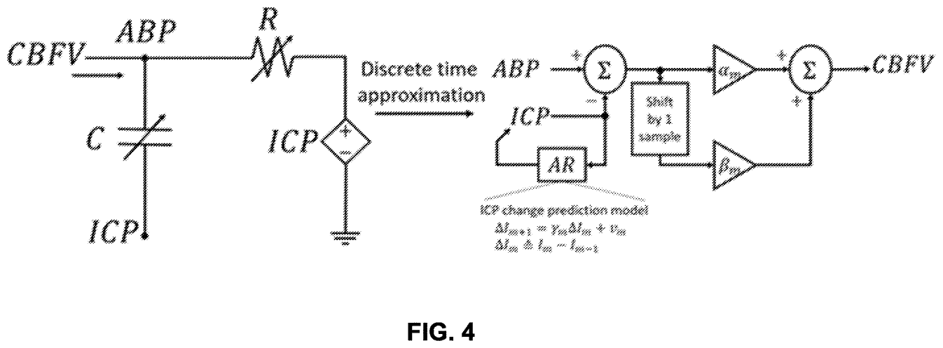

The statistical model, which may be used by both ICP estimation technique 108 and ICP change tracking technique 112, may relate arterial blood pressure and cerebral blood flow velocity to intracranial pressure using a physiological model. Parameters of the statistical model may represent different physiological characteristics. In some embodiments, the statistical model may include one or more parameters representing resistance, cerebrovascular compliance, inertance, and intracranial pressure where arterial blood pressure and cerebral blood flow velocity are inputs to the statistical model. FIG. 4 is a diagram of a statistical model, which may be used in estimating intracranial pressure according to some embodiments. The circuit diagram on the left is representative of a physiological model relating resistance (R), cerebrovascular compliance (C), and intracranial pressure (ICP) with arterial blood pressure (ABP) and cerebral blood flow velocity (CBFV). The statistical model may adopt a discretized time approximation form of this physiological model for estimating ICP values. In some embodiments, the statistical model may be implemented using Bayesian statistics. Additional details relating to this physiological model shown on the left-hand side in FIG. 4 and how it can be used in calculating intracranial pressure may be found in U.S. Pat. No. 8,366,627, issued on Feb. 5, 2013, which is incorporated by reference in its entirety. Additional discussion on a statistical model used in estimating intracranial pressure is described herein including in Sections A.2.1, A.2.2, and A.4.2.

ICP estimation technique 108 and ICP change tracking technique 112 may involve using Bayesian statistical techniques to compute ICP baseline value 110 and change in ICP value(s) 114, respectively. In some embodiments, ICP estimation technique 108 may involve using a statistical model to compute a posterior distribution of ICP values based on patient data 102 and a prior distribution of ICP values corresponding to data obtained from one or more other people. In some embodiments, the prior distribution of ICP values may be obtained by directly measuring ICP in patients using invasive techniques. In some embodiments, ICP change tracking technique 112 may involve using a statistical model to compute a posterior distribution of ICP values based on patient data 102 and a prior distribution of ICP values, which may be a uniform distribution according to some embodiments.

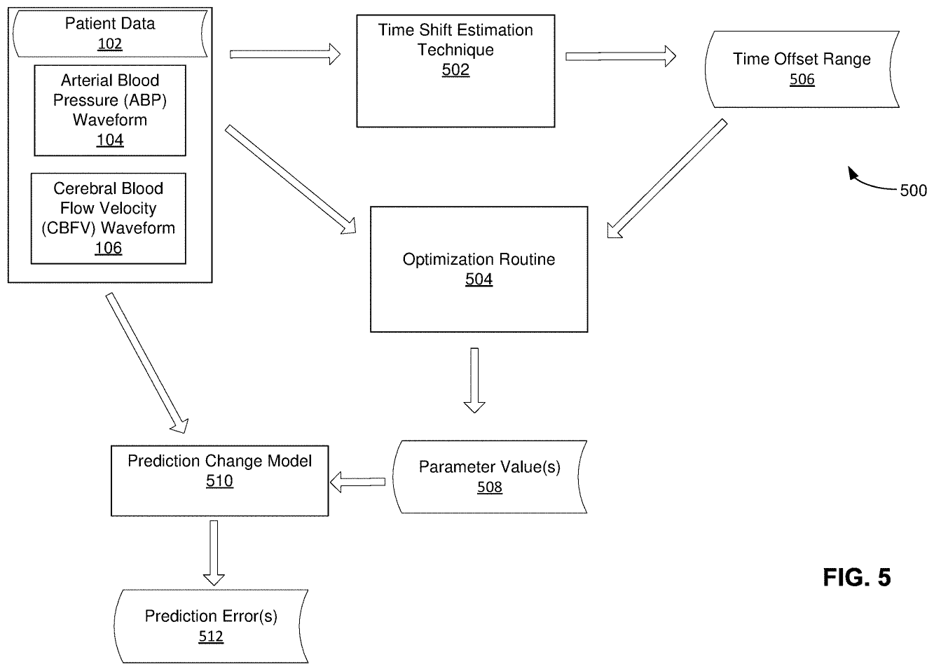

Some embodiments of the statistical model used by ICP estimation technique 108 and ICP change tracking technique 112 may involve estimating time shifts between ABP and CBFV waveforms and using those estimated time shifts in optimizing parameters of the statistical model. FIG. 5 is a diagram of an illustrative processing pipeline 500 for optimizing parameters of a statistical model, which may be implemented as part of ICP estimation technique 108 and/or ICP change tracking technique 112, in accordance with some embodiments of the technology described herein.

Time shift estimation technique 502 may be used to estimate time offset range 506 using ABP waveform 104 and CBFV waveform 106. In some embodiments, a statistical model, which may be used by ICP estimation technique 108 and ICP change tracking technique 112, may involve aligning in time ABP waveform 104 and CBFV waveform 106 using time offset range 506 estimated by time shift estimation technique 502. Time offset range 506 may include at least one time offset value that may act to shift ABP waveform 104 and CBFV waveform 106 into an alignment meeting a set of physiological constraints. Time shift estimation technique 502 may involve determining one or more alignments in time between ABP waveform 104 and CBFV waveform 106 such that one or more constraints in the set of constraints is met for at least one cardiac cycle. According to some embodiments, time shift estimation technique 502 may involve selecting one or more time offset values from a set of possible time offset values based on the alignment of ABP waveform 104 and CBFV waveform 106 meeting the set of physiological constraints. In some embodiments, the set of constraints may include constraining the alignment of ABP waveform 104 and CBFV waveform 106 such that a systolic peak in CBFV occurs prior to a systolic peak in ABP. In some embodiments, the set of constraints may include constraining the alignment of ABP waveform 104 and CBFV waveform 106 such that a diastolic point in CBFV occurs at substantially the same time as a diastolic point in ABP. Additional discussion for time-aligning ABP and CBFV waveforms is described herein including in Section A.4.3.

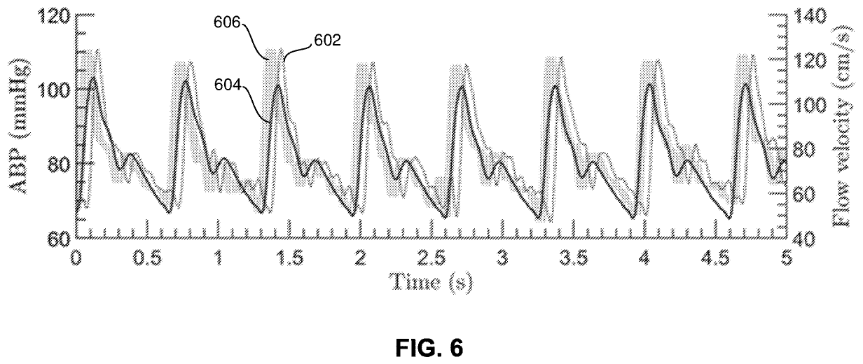

FIG. 6 is an exemplary plot of illustrating an arterial blood pressure (ABP) waveform 604 (shown by the solid line), cerebral blood flow velocity (CBFV) waveform 602 (shown by the dotted line), and shifted CBFV waveforms 606 (shown by the grey band). Shifted CBFV waveforms 606 have been identified by shifting CBFV waveform 602 by a range of time shifts, which may be determined using time shift estimation technique 502. As shown in FIG. 6, some or all of the shifted CBFV waveforms 606 may meet one or more physiological constraints. For example, at least some of the shifted CBFV waveforms 606 have systolic peaks that occur prior to systolic peaks in ABP waveform 604. As another example, at least some of the shifted CBFV waveforms 606 have diastolic points that occur at substantially the same time as the diastolic peaks in ABP waveform 604.

Optimization routine 504 may be used to determine parameter value(s) 508 of a statistical model used by ICP estimation technique 108 and ICP change tracking technique 112. In particular, optimization routine 504 may determine parameter value(s) 508 by using patient data 102 and time offset range 505. In some embodiments, optimization routine 504 may involve evaluating multiple ICP values at different time offsets in time offset range 506. Optimization routine 504 may involve evaluating different pairs of an ICP value and a time offset using the ABP waveform 104 and CBFV waveform 106. In some embodiments, evaluating different pairs of an ICP value and a time offset value may involve performing parallel computational processing of the different pairs. Such parallel processing may allow for improved computational efficiency in estimating intracranial pressure, particularly during real-time monitoring of intracranial pressure. Optimization routine 504 may be performed by implementing any suitable statistical techniques, including a regularized least squared error estimation, a constrained error estimation, and/or an unconstrained error estimation. Additional discussion for performing an optimization routine to determine model parameters is described herein including in Section A.4.3.

Prediction change model 510 may be used to predict physiological signal(s) (e.g., ABP, CBFV) using patient data 102 and the statistical model implemented by ICP estimation technique 108 and ICP change tracking technique 112. As shown in FIG. 5, prediction change model 510 may predict prediction error(s) 512 for the predicted physiological signal(s) using parameter value(s) 508 determined by optimization routine 504. In some embodiments, prediction error(s) 512 may be generated by comparing the predicted physiological signal(s) to patient data 102.

Some embodiments involve using one physiological signal to predict a different physiological signal. In some embodiments, optimization routine 504 may involve using ABP waveform 104 to evaluate different pairs of an ICP value and a time offset value to determine parameter value(s) 508. In such embodiments, prediction change model 510 may be used to predict a CBFV waveform for a future time period and generate prediction error(s) 512 based on comparing the predicted CBFV waveform for the future time period and a portion of CBFV waveform 106 corresponding to that time period. In other embodiments, optimization routine 504 may involve using CBFV waveform 106 to evaluate different pairs of ICP values and time offset values to determine parameter value(s) 508. In such embodiments, prediction change model 510 may be used to predict an ABP waveform for a future time period and generate prediction error(s) 512 based on comparing the predicted ABP waveform for the future time period and a portion of ABP waveform 104 corresponding to that time period.

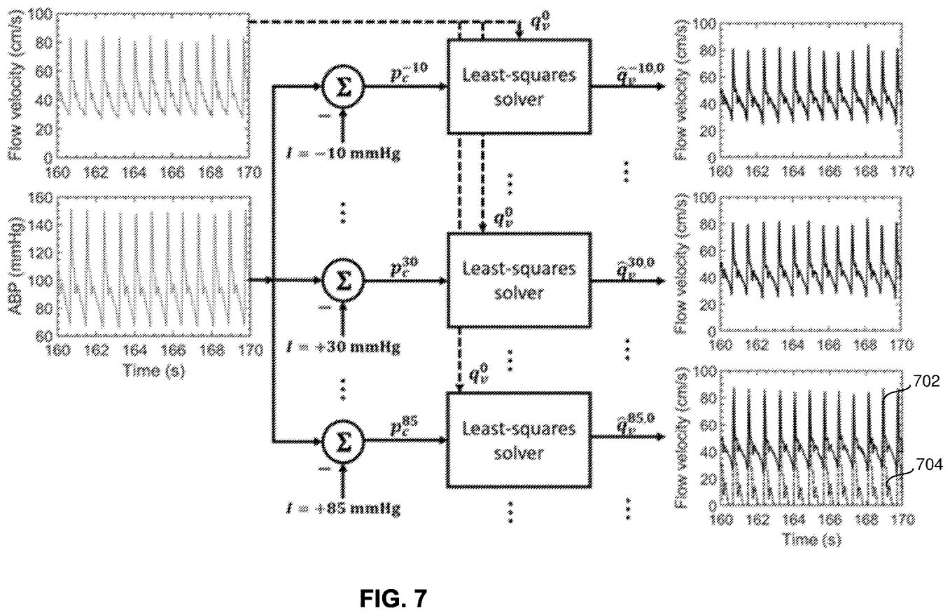

FIG. 7 is diagram of an optimization routine used in estimating intracranial pressure, which may be used in accordance with some embodiments of the technology described herein. As shown in FIG. 7, ABP waveform is used by optimization routine to evaluate different pairs of ICP values (−10 mmHg, +30 mmHg, and +85 mmHg) and different time offsets (d). The parameter value(s) determined by this optimization process may be used in predicting CBFV waveforms for each of the different ICP values. FIG. 7 shows a predicted CBFV waveforms for each of the different ICP values and the observed CBFV waveforms obtained from patient data for the corresponding time period. Prediction errors can be determined by comparing the predicted CBFV waveform for each of the different ICP values with its corresponding observed CBFV waveform. As an example, the predicted CBFV waveform 704 for ICP value at +85 mmHg in comparison to the observed CBFV waveform 702 which is obtained from the patient data. Since predicted CBFV waveform 704 differs from the observed CBFV waveform 702, then prediction errors generated from comparing predicted CBFV waveform 704 with observed CBFV waveform 702 may indicate there being a low level of accuracy in using the parameter values obtain from performing the optimization routine with an ICP value of +85 mmHg. For the other ICP values shown in FIG. 7, −10 mmHg and +30 mmHG, the predicted CBFV waveform is more similar to the observed CBFV waveform than for predicted CBFV waveform 704, which may indicate a high level of accuracy in using the parameter values obtained from performing the optimization routine using these ICP values.

Estimating an ICP value may involve using a Bayesian statistical framework. Accordingly, some embodiments may involve using a statistical model to compute a posterior distribution of ICP values based on a likelihood of ICP given patient data and a prior distribution of ICP values. Some embodiments include using prediction error(s) 512 obtained from using prediction change model 510 in determining the likelihood of ICP. In some embodiments, computing the likelihood of ICP may involve determining a likelihood of ICP for the different time offsets and ICP values used in the processing performed by optimization routine 504 where the likelihood of ICP is computed using prediction error(s) 512 generated by prediction change model 510. In some embodiments, different likelihood of ICP distributions may be obtained for different time offsets and a single likelihood of ICP distribution may be determined by combining the different likelihood of ICP distributions. In this manner, the likelihood of ICP that may be generated using prediction error(s) 512 may collapse onto one-dimension (e.g., ICP). Combining the different likelihood of ICP distributions may involve using any suitable statistical methods, including averaging across all distributions (e.g., marginalization methods) and selecting the highest likelihood ICP value (e.g., likelihood maximization methods).

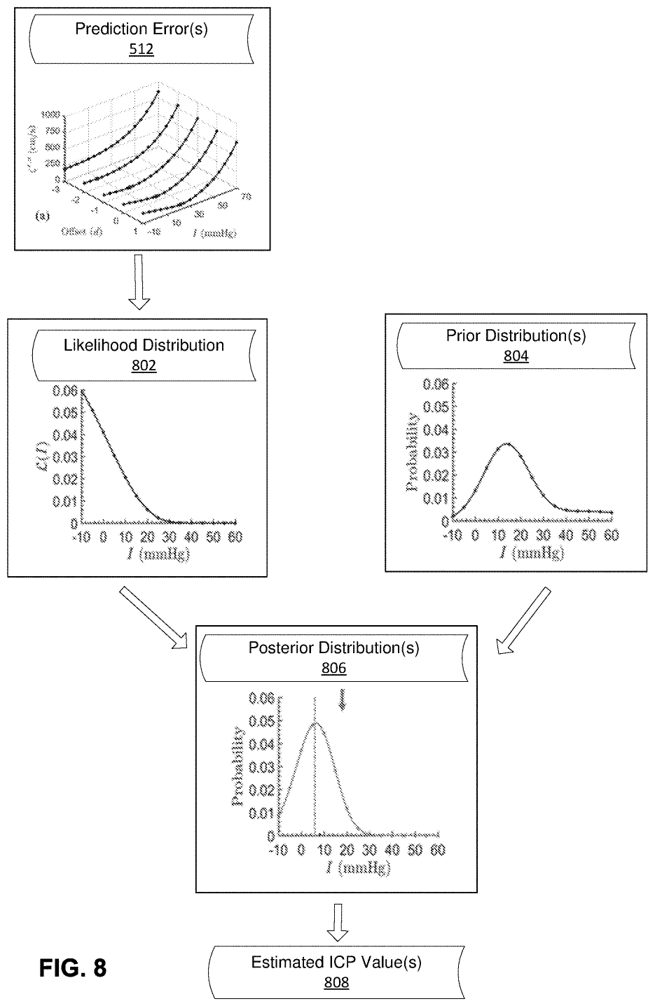

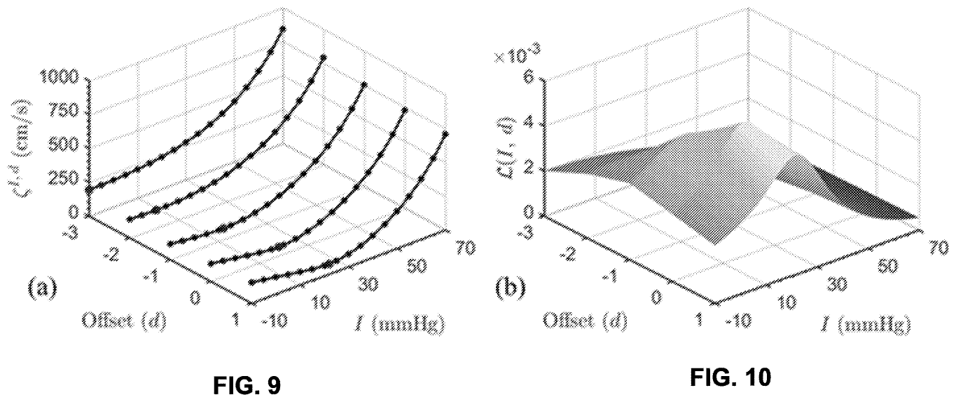

FIG. 8 is a diagram of an illustrative data processing pipeline for estimating intracranial pressure using prediction errors. As shown in FIG. 8, prediction error(s) 512, which may be obtained from optimization routine 504, may be used in determining likelihood distribution of ICP 802. Prediction error(s) 410 (0 may be found for each combination of time offset (d) and ICP value (I) used by optimization routine 504. FIG. 9 is an exemplary plot illustrating prediction errors versus time offsets and intracranial pressure. FIG. 10 is plot illustrating a likelihood distribution across both time offsets (d) and ICP values (I) corresponding to the prediction error(s) shown in FIG. 9. Likelihood distribution of ICP 802 may be obtained based on prediction error(s) by collapsing the likelihood distributions along the ICP dimension.

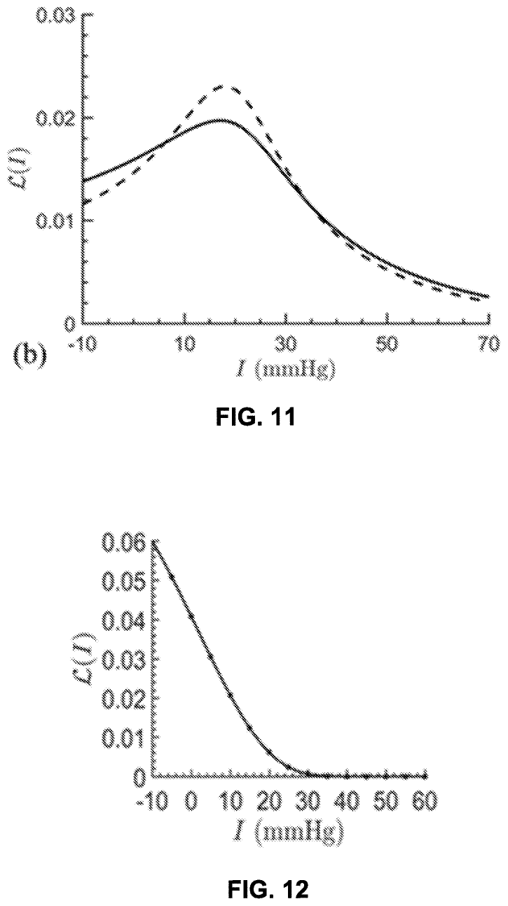

In some embodiments, a likelihood distribution of ICP may be obtained by relating the prediction errors to the likelihood using an exponential relationship, such as in the following equation:

$$ℒ ( I , d ) = \frac{1}{S} \times exp \{ - {( \frac{{ζ}^{I , d}}{m} )}^{p} \} where m = {min}_{I , d} {ζ}^{I , d}$$

where ζ^{I,d} is the prediction errors, and S is chosen so that [symbol] (I, d) sums to unity. FIG. 11 is an exemplary plot of likelihood of intracranial pressure versus intracranial pressure where an exponential relationship between likelihood distribution of ICP and prediction errors is implemented.

In some embodiments, a likelihood distribution of ICP may be obtained by relating the prediction errors to the likelihood using an inverse relationship, such as in the following equation:

$$ℒ ( I , d ) = \frac{1}{S} \times \frac{1}{{ζ}^{I , d}}$$

FIG. 12 is another exemplary plot of likelihood of intracranial pressure versus intracranial pressure where an inverse relationship between likelihood distribution of ICP and prediction errors is implemented.

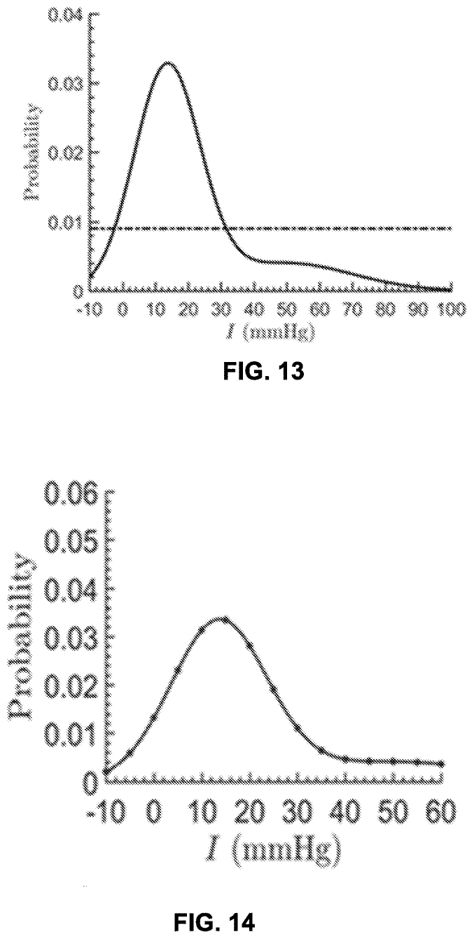

As shown in FIG. 8, prior distribution 804 and likelihood distribution 802 may be combined to obtain posterior distribution 806. FIGS. 13 and 14 are exemplary plots of prior distribution of intracranial pressure versus intracranial pressure. In some embodiments, a prior distribution may be obtained by fitting one or more Gaussian models to a set of ICP data, such as data obtained from patients using invasive techniques. Such a prior distribution may be used in estimating an initial ICP value, such as by ICP estimation technique 108. In some embodiments, a prior distribution may be a uniform distribution, which may not be dependent on any patient data. Such a prior distribution may be used in tracking change in ICP, such as by ICP change tracking technique 112.

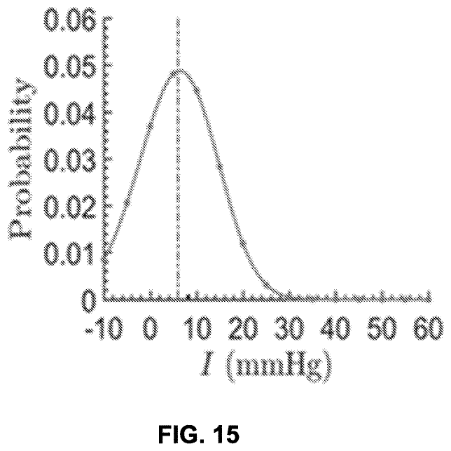

Posterior distribution 806 may be obtained by combining likelihood distribution 802 and prior distribution 804, such as by performing a pointwise multiplication of probabilities. FIG. 15 is an exemplary plot of a posterior distribution of intracranial pressure versus intracranial pressure. Estimated ICP value(s) 808 may be determined from posterior distribution 806. Posterior distribution 806 may be used in providing an estimate for an ICP value using any suitable statistical technique, including obtaining the mean, mode, or median of the posterior distribution. Additional discussion for estimating ICP values by using a likelihood distribution and prior distribution to obtain a posterior distribution is described herein including in Section A.4.3.

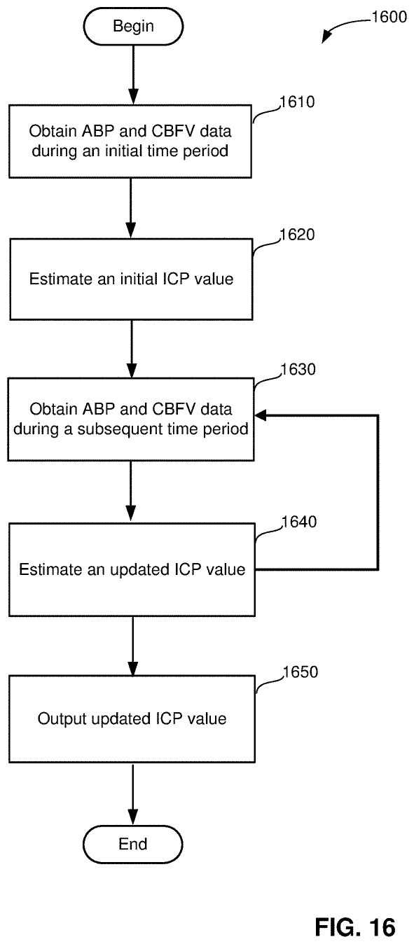

FIG. 16 is a flow chart of an illustrative process 1600 for estimating intracranial pressure, in accordance with some embodiments of the technology described herein. Process 1600 may be performed on any suitable computing device(s) (e.g., a single computing device, multiple computing devices co-located in a single physical location or located in multiple physical locations remote from one another, one or more computing devices part of a cloud computing system, etc.), as aspects of the technology described herein are not limited in this respect. In some embodiments, ICP pressure estimation technique 108 and ICP change tracking technique 112 may perform some or all of process 1600 to estimate ICP for a patient.

Process 1600 begins at act 1610, where data identifying arterial blood pressure (ABP) and cerebral blood flow velocity (CBFV) from a patient during an initial time period is obtained. The ABP and CBFV data are obtained at different locations of the patient. Obtaining the data include obtaining ABP and CBFV of the patient over multiple of cardiac cycles to obtain ABP and CBFV waveforms.

Next, process 1600 proceeds to act 1620, where an initial ICP value is estimated, such as by using ICP estimation technique 108. In some embodiments, estimating an initial ICP value involves using a statistical model to compute a posterior distribution of ICP values based on the data and a prior distribution of intracranial pressure values, which in some embodiments may correspond to data obtained from at least one person other than the patient. In some embodiments, estimating the initial ICP value may involve using the statistical model to compute the posterior distribution of ICP values based on a likelihood of ICP given the data and the prior distribution. The statistical model may relate ABP and CBFV to ICP, and in some embodiments may include one or more parameters representing physiological characteristics (e.g., cerebrovascular resistance, cerebrovascular compliance, and cerebrovascular inertance). In some embodiments, the statistical model includes a parameter representing cerebrovascular resistance, a parameter representing cerebrovascular compliance, and a parameter representing intracranial pressure.

Next process 1600 proceeds to act 1630, data identifying arterial blood pressure (ABP) and cerebral blood flow velocity (CBFV) from a patient during a subsequent time period is obtained. The ABP and CBFV data are obtained at different locations of the patient. Obtaining the data include obtaining ABP and CBFV of the patient over multiple of cardiac cycles to obtain ABP and CBFV waveforms.

Next process 1600 proceeds to act 1640, where an updated ICP value is estimated, such as by using ICP change tracking technique 112 to estimate at least one change in ICP value to combine with the initial ICP value estimated in step 1620. Estimating the updated ICP value may involve determining a change in ICP of the patient based on the data and the initial ICP value. In some embodiments, determining the change in ICP is performed at least in part by using the statistical model and the data to estimate one or more values for parameter(s) of the statistical model. Next process 1600 proceeds to act 1650, where an indication of the updated ICP value is output, such as to a user via a user interface.

Some embodiments involve estimating a series of ICP values for a patient by repeating act 1630 and act 1640 as additional patient data is obtained, which may allow for real-time monitoring of ICP in the patient. Estimating the series of ICP values for the patient involve determining changes in ICP of the patient based on the additional data and combining the changes in ICP with the initial intracranial pressure value. In some embodiments, estimating the series of ICP values may include dynamically updating an ICP value during subsequent time periods. The dynamic updating of the ICP value may be performed in an adaptive manner.

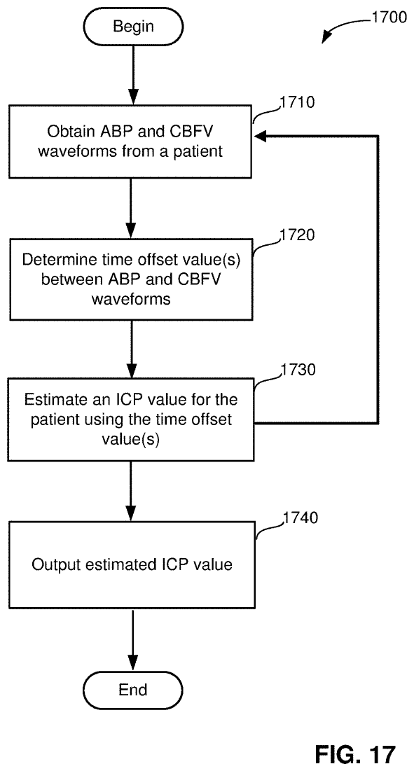

FIG. 17 is a flow chart of an illustrative process 1700 for estimating intracranial pressure, in accordance with some embodiments of the technology described herein. Process 1700 may be performed on any suitable computing device(s) (e.g., a single computing device, multiple computing devices co-located in a single physical location or located in multiple physical locations remote from one another, one or more computing devices part of a cloud computing system, etc.), as aspects of the technology described herein are not limited in this respect. In some embodiments, ICP pressure estimation technique 108, ICP change tracking technique 112, and time shift estimation technique 502 may perform some or all of process 1700 to predict chemical reaction(s) and output molecule(s).

Process 1700 begins at act 1710, where data identifying arterial blood pressure (ABP) and cerebral blood flow velocity (CBFV) waveforms from a patient is obtained. The ABP and CBFV data are obtained at different locations of the patient. Obtaining the data include obtaining ABP and CBFV of the patient over multiple of cardiac cycles to obtain ABP and CBFV waveforms.

Next, process 1700 proceeds to act 1720, where time offset value(s) between ABP and CBFV waveforms are determined, such as by using time shift estimation technique 502. In some embodiments, determining the time offset value(s) involve aligning in time the ABP and CBFV waveforms. Aligning the ABP and CBFV waveforms may include constraining the alignment, for at least one cardiac cycle, such that a systolic peak in cerebral blood flow velocity occurs prior to a systolic peak in arterial blood pressure. Aligning the ABP and CBFV waveforms may include constraining the alignment, for at least one cardiac cycle, such that a diastolic point in cerebral blood flow velocity occurs at substantially the same time as a diastolic point in arterial blood pressure. Some embodiments involve selecting the time offset value(s) from multiple time offset values based on the alignment of the arterial blood pressure waveform and the cerebral blood flow velocity waveform meeting a set of physiological constraints (e.g., a systolic peak in cerebral blood flow velocity occurs prior to a systolic peak in arterial blood pressure, a diastolic point in cerebral blood flow velocity occurs at substantially the same time as a diastolic point in arterial blood pressure).

Next, process 1700 proceeds to act 1730, where an ICP value for the patient is estimated using the time offset value(s), such as by using ICP pressure estimation technique 108 and/or ICP change tracking technique 112. Estimating the ICP value may involve using a statistical model to compute a posterior distribution of ICP values based on a likelihood of intracranial pressure given the data and a prior distribution of ICP values. In some embodiments, the prior distribution of ICP values may correspond to data obtained from at least one person other than the patient. Next process 1700 proceeds to act 1740, where an indication of the estimated ICP value is output, such as to a user via a user interface.

Some embodiments involve estimating a series of ICP values for a patient by repeating acts 1710, 1720, and 1730 as additional patient data is obtained, which may allow for real-time monitoring of ICP in the patient. Estimating the series of ICP values for the patient involve determining changes in ICP of the patient based on the additional data and combining the changes in ICP with the initial intracranial pressure value. In some embodiments, estimating the series of ICP values may include dynamically updating an ICP value during subsequent time periods.

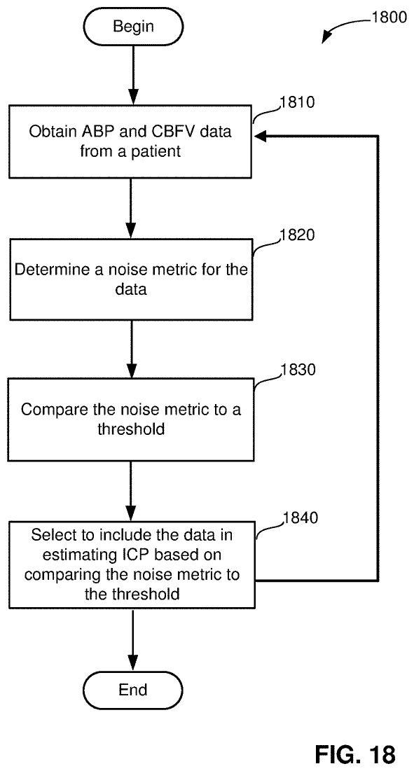

FIG. 18 is a flow chart of an illustrative process 1800 for evaluating noise in patient data, in accordance with some embodiments of the technology described herein. Process 1800 may be performed on any suitable computing device(s) (e.g., a single computing device, multiple computing devices co-located in a single physical location or located in multiple physical locations remote from one another, one or more computing devices part of a cloud computing system, etc.), as aspects of the technology described herein are not limited in this respect. In some embodiments, ICP estimation technique 108 and/or ICP change tracking technique 112 may perform some or all of process 1800 to evaluate noise in patient data as part of determining which patient data to include in estimating an ICP value.

Process 1800 begins at act 1810, where data identifying arterial blood pressure (ABP) and cerebral blood flow velocity (CBFV) data from a patient is obtained over a time period. Next, process 1800 proceeds to act 1820, where a noise metric for the data, where the metric indicates a level of noise in the data during the time period. In some embodiments, the noise metric may be determined by comparing the ABP and CBFV data to determine a level of similarity between the ABP and CBFV waveforms. Some embodiments involve computing a cross-correlation of the ABP and CBFV waveforms where an output of the cross-correlation indicates a level of similarity between the ABP and CBFV waveforms. ABP and CBFV waveforms that have substantially similar profiles will have a noise metric indicating a low level of noise in the ABP and CBFV waveforms. ABP and CBFV waveforms that have dissimilar profiles will have a noise metric indicating a high level of noise in the ABP and CBFV waveforms.

Next, process 1800 proceeds to act 1830, where the noise metric is compared to a threshold, and to act 1840, where the data is selected to include in estimating ICP based on the comparison of the noise metric to the threshold. In some embodiments, if the noise metric is less than the threshold, then the data associated with the time period is indicated as having a low noise level and is included in estimating ICP. In some embodiments, if the noise metric is more than the threshold, then the data is indicated as having a high noise level and is not included in estimating ICP. Process 1800 may be repeated for individual time periods, such as in response to receiving additional patient data. In some embodiments, process 1800 is performed on different time periods for patient data used by ICP estimation technique 108 and/or ICP change tracking technique 112.

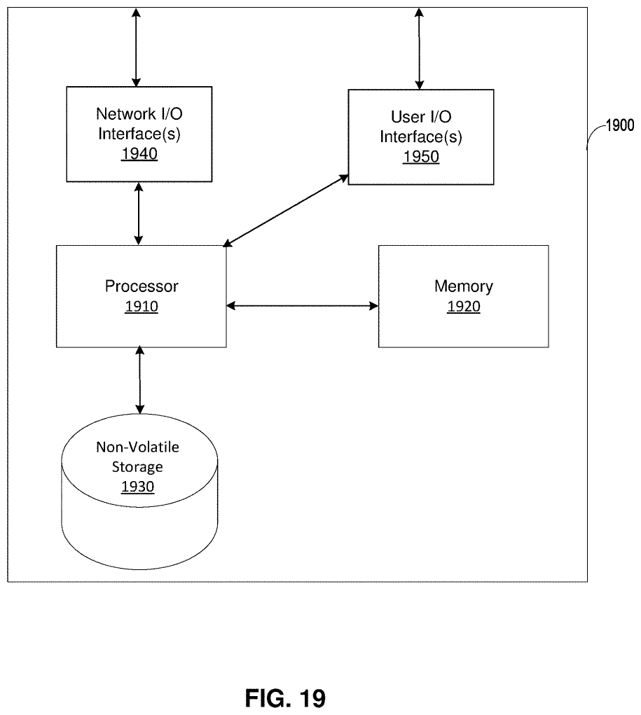

An illustrative implementation of a computer system 1900 that may be used in connection with any of the embodiments of the technology described herein is shown in FIG. 19. The computer system 1900 includes one or more processors 1910 and one or more articles of manufacture that comprise non-transitory computer-readable storage media (e.g., memory 1920 and one or more non-volatile storage media 1930). The processor 1910 may control writing data to and reading data from the memory 1920 and the non-volatile storage device 1930 in any suitable manner, as the aspects of the technology described herein are not limited in this respect. To perform any of the functionality described herein, the processor 1910 may execute one or more processor-executable instructions stored in one or more non-transitory computer-readable storage media (e.g., the memory 1920), which may serve as non-transitory computer-readable storage media storing processor-executable instructions for execution by the processor 1910.

Computing device 1900 may also include a network input/output (I/O) interface 1940 via which the computing device may communicate with other computing devices (e.g., over a network), and may also include one or more user I/O interfaces 1950, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

The above-described embodiments can be implemented in any of numerous ways. For example, the embodiments may be implemented using hardware, software or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-discussed functions of one or more embodiments. The computer-readable medium may be transportable such that the program stored thereon can be loaded onto any computing device to implement aspects of the techniques discussed herein. In addition, it should be appreciated that the reference to a computer program which, when executed, performs any of the above-discussed functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

Some aspects of the technology described herein may be understood further based on the non-limiting illustrative embodiments described below in Section A. Any limitations of the embodiments described below in Section A are limitations only of the embodiments described in Section A, and are not limitations of any other embodiments described herein.

Section A

A noninvasive intracranial pressure (ICP) estimation method is proposed that incorporates model-based estimation within a probabilistic framework. A first-order subject-specific model of the cerebral vasculature relates arterial blood pressure with cerebral blood flow velocity. The model is solved for a range of physiologically plausible mean ICP values, and the resulting residual errors are transformed into likelihoods for each candidate ICP. First, a baseline ICP estimate is established by combining the likelihoods with a multi-modal prior distribution of the ICP to yield an a posteriori distribution whose mode is taken as the baseline ICP estimate. A single-state model of cerebral autoregulatory dynamics is then employed in subsequent data windows to track changes in the baseline by combining ICP estimates obtained with a uniform prior belief and model-predicted ICPs. The method yielded an ICP estimation bias (mean error or accuracy) of 0.6 mmHg and a root-mean-squared error (or precision) of 4.2 mmHg on data from thirteen patients at Boston Children's Hospital. These performance characteristics are well within the acceptable range for clinical decision making. The method proposed here therefore constitutes a significant step towards robust, continuous, patient-specific noninvasive ICP determination.

1. Introduction

Intracranial pressure (ICP) is the hydrostatic pressure of cerebrospinal fluid (CSF), the fluid that surrounds and cushions human brain tissue. Elevated ICP hampers brain tissue perfusion, and can lead to severe cerebral ischemic injury. Such elevations can occur in neuropathological conditions that include hydrocephalus, traumatic brain injury (TBI), hemorrhagic stroke, and brain tumors. Severe TBI, for example, is estimated to cause 52,000 deaths annually in the United States. TBI management requires accurate ICP measurement, as does hydrocephalus care, which is estimated to incur over US$1 billion annually in the U.S.

The normal mean ICP range in healthy adults in the supine posture is reported to range from 6 to 18 mmHg. In children, normal mean ICP to may range from 8 to 21 mmHg. ICP elevations beyond this normal range are lowered aggressively in current clinical practice. The latest guidelines for TBI care, for instance, recommend maintaining mean ICP of less than 22 mmHg.

Clinical ICP measurement modalities are invasive, require neurosurgical expertise, and carry an associated risk of infection. ICP measurement is therefore used only for severely ill patients, despite the fact that a larger pool of subjects may otherwise benefit from direct ICP measurement. This potential need has prompted the development of noninvasive ICP (nICP) estimation schemes. Examples of nICP estimation methods include applying external pressure on the eyeball to balance retro-orbital pressure with ICP, measuring cerebral blood flow velocity (CBFV) indices, and exploiting transcranial acoustic signal properties. Tympanic membrane displacement, and optic nerve sheath distension have also been shown to correlate with ICP. Physiologic model-based methods have been proposed, along with statistical learning frameworks. Despite these efforts, reliable and continuous nICP estimation has remained elusive and has not been adopted in clinical practice.

In this paper, we present a robust physiologic nICP estimation and tracking scheme. We model cerebral hemodynamics with a first-order, time-varying, finite impulse response (FIR) filter that relates cerebral arterial blood pressure (cABP), ICP, and CBFV. This model incorporates a first-order autoregressive (AR) process description of ICP dynamics. We use CBFV measured via transcranial Doppler (TCD) ultrasonography and radial arterial blood pressure (rABP). An associated Bayesian estimation framework is proposed to compute nICP estimates that are robust against both morphological differences between rABP and cABP, and physiologically-induced time offsets between rABP and CBFV. In this Bayesian framework, we solve our model for a physiologically plausible range of candidate ICPs and time offsets to form an ICP likelihood distribution. We combine this distribution with a preset multi-modal prior belief about the patient's ICP, and select the resulting posterior distribution's mode as the baseline ICP. Subsequent changes in the ICP are computed with a uniform prior belief to reduce dependence on the initial prior distribution. The estimated ICP changes are filtered via predictions obtained from the AR model of ICP dynamics for increased robustness.

2. Results

2.1 Model of Cerebral Hemodynamics

Sophisticated multi-parameter models that describe complex cerebrovascular behaviors are not suited for nICP estimation because their parameters are difficult to identify in a simple, noninvasive, robust, and patient-specific manner. Our group has previously proposed a Windkessel-like model that relates cerebral perfusion pressure (CPP), the difference between cABP and ICP, with cerebral blood flow, and hence CBFV. This model represents cerebrovascular blood flow resistance and vascular and brain tissue compliance with a variable resistor, R, and capacitor, C, respectively. The model, however, does not describe temporal evolution of the ICP; it is used to estimate mean ICP for each window of ABP and CBFV data segments.

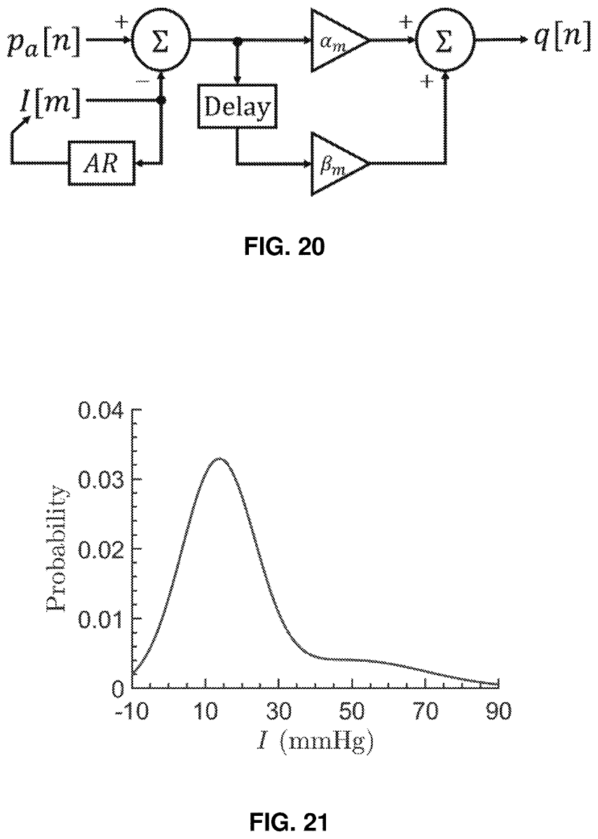

Here, we have used a time-varying, first-order FIR filter approximation of our previous model with the addition of an AR process description of ICP dynamics. A first-order approximation was chosen because cABP and CBFV are quasi-periodic signals, and their spectral content is concentrated around a few frequency harmonics, limiting the order of models whose parameters can be reliably estimated using only the cABP and CBFV. As shown in the Methods section, the FIR filter coefficients are functions of R and C, and are assumed to remain constant during estimation windows comprising twenty cardiac cycles. This is because modulations in R and C are assumed to occur over longer timescales. Likewise, ICP is also considered to be a constant during an estimation window. The resulting model is shown in FIG. 20, and can be mathematically described as

q[n]=αₘ(pₐ[n]−I[m])+βₘ(pₐ[n−1]−I[m]) (1)

where q and pₐ denote the CBFV and cABP, respectively, and sampling and estimation window indices are denoted by n and m, respectively. The filter taps, αₘ and βₘ, and the mean ICP, I[m], are assumed to remain constant during individual estimation windows. Temporal evolution of the ICP is modeled by a first-order AR model which of the form

ΔI[m+1]=γₘΔI[m]+vₘ (2)

where ΔI[m]=I[m]−I [m−1], is the inter-estimation-window ICP change, γₘ is a parameter that represents the autoregulatory state, and vₘ is a white-noise sequence with variance σₘ_{ᵥ}². For model stability |γₘ|<1 is chosen. A value of |γₘ| close to +1 models the tendency of ICP to rise or fall rapidly, whereas a negative γₘ models static ICPs that only vary slightly about their baseline. This AR model can be used to predict future changes in ICP, which can then be used to refine subsequent nICP estimates.

2.2 Model-Based Estimation Algorithm

CBFV and cABP recordings can be used to estimate the ICP using our model. In practice, however, cABP recordings are not available in clinical settings, and thus we use rABP instead. The human blood pressure profile changes along the arterial tree due to reflections from arterial branching sites and vessel taper. There is also a physiologically induced time delay between rABP and cABP due to finite wave propagation velocities. These together can introduce errors in the estimated nICPs. Hence, we developed a probabilistic estimation framework to reduce sensitivity of our nICP estimates on these factors.

In our method, we first establish a baseline ICP and subsequently track changes in this baseline. The baseline is determined by fitting the model to measured rABP and CBFV for a range of physiologically plausible ICP values and time offsets between rABP and CBFV. The fitting is achieved a least-squared-error sense. The residual errors are then transformed into a likelihood distribution of ICP values. This likelihood is combined with a preset prior distribution. The mode of the resulting a posteriori distribution is taken as the nICP estimate. This procedure is repeated for several windows, and the nICP estimates are averaged together to yield the baseline. The prior distribution employed in this stage generously models ICP values encountered at the bedside—extremely high and low values are given significant weight—in order to ensure our method's generalizability. The distribution is shown in FIG. 21, and its construction process is outlined in detail in the Methods section.

After this initial baseline estimation stage, ICP estimates are computed with a uniform distribution to reduce dependence on the initial prior belief. A downside of using a uniform distribution, however, is that the resulting nICP estimates are more error-prone than before. In our method, we addressed this problem by filtering changes in estimated nICPs by model-predicted ICP changes via a Kalman filter-like approach, and subsequently adding the filtered ICP changes back to the baseline.

2.3 Data Description and Method Validation

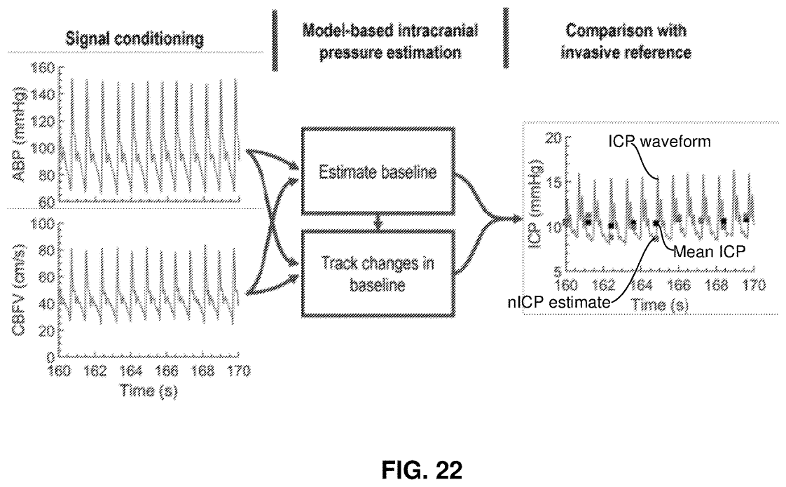

We used data that were collected at Boston Children's Hospital (BCH) between February 2015 and June 2017. The data collection protocols were approved by the relevant Institutional Review Boards at BCH and MIT, and informed consent was obtained from patients or their surrogates prior to data collection. Individual recording sessions lasted for nearly twenty minutes during which the rABP, CBFV, and (invasive) ICP waveforms were recorded simultaneously. Important metadata including height differences between the location of ICP and rABP transducers were also recorded. Data were collected from thirteen patients suffering from diverse pathologies that included TBI, hydrocephalus, and hemorrhagic strokes of various types. We tested our method's performance on noise-free data segments (Table 1) extracted from the ensemble data. As illustrated in FIG. 22, the extracted rABP and CBFV signals were passed through a signal conditioning stage. The conditioned data were then passed to the estimation routine that computed nICP estimates in non-overlapping twenty-cardiac-beat windows. The nICP estimates were then compared with the reference ICP measurements.

TABLE 1
Patient Information

| | | Age | | Min. | Recording | Duration | [Min., Max.] |
|---|---|---|---|---|---|---|---|
| Subject | Gender | (Yrs) | Diagnosis | GCS | sessions | (hr:min) | ICP (mmHg) |
| 1 | M | 12 | Stroke | 6 | 11 | 2:02 | [9, 22] |
| 2 | F | 16 | Traumatic brain injury | 4 | 6 | 0:44 | [5, 9] |
| 3 | M | 14 | Stroke | — | 2 | 0:21 | [7, 12] |
| 4 | F | 2 | Hemorrhage | 3 | 2 | 0:25 | [8, 16] |
| 5 | F | 11 | Brain tumor | 3 | 3 | 0:09 | [5, 11] |
| 6 | M | 18 | Intraventricular hemorrhage | 14 | 4 | 0:26 | [8, 25] |
| 7 | M | 20 | Hydrocephalus | 3 | 3 | 0:27 | [8, 20] |
| 8 | M | 11 | Traumatic brain injury | — | 1 | 0:08 | [6, 14] |
| 9 | M | 7 | Traumatic brain injury | 3 | 4 | 0:41 | [4, 21] |
| 10 | F | 6 | Hydranecphaly/Hydropcephalus | — | 1 | 0:01 | [7, 7] |
| 11 | M | 4 | Cerebrohepatopathy | — | 2 | 0:34 | [1, 16] |
| 12 | M | 6 | Cavernous malformation | 15 | 2 | 0:31 | [4, 8] |
| 13 | M | 25 | Chiari malformation | — | 1 | 0:10 | [4, 13] |
| Ensemble | 4 F, 9 M | 2-25 | | 3 15 | 42 | 6:40 | [1, 25] |

2.4 ICP Estimation Results

Nearly seven hours of data (1657 estimation windows from 118 data records) were analyzed, and estimates were computed in a fully automated manner for reproducibility. The following results were computed by setting γₘ=0.8, with σₘ_{ᵥ}²=49 mmHg² in Equation 2 to model rapidly changing ICPs. These parameters help ensure our method's generalizability to diverse datasets, and were determined during a pilot exploration on data subsets from three patients.

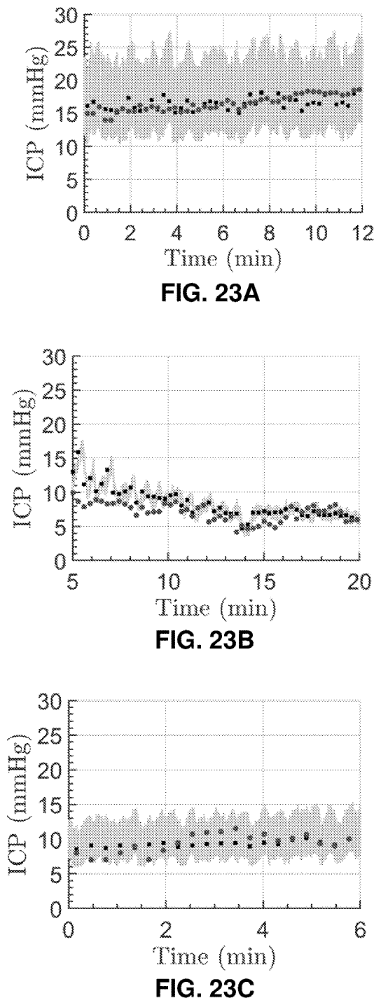

Examples of the estimation results are shown in FIGS. 23A, 23B, and 23C. The first recording is from a stroke patient (Patient 1). The nICP estimation bias in this case was 0.0 mmHg with an RMSE of 1.1 mmHg. The second recording is from another stroke patient (Patient 3). The estimation bias in this case was −1.4 mmHg with an RMSE of 4.7 mmHg. In this case, CSF was being actively drained, and the ICP pulsatility was small. The third recording is from a patient suffering from cerebrohepatopathy (Patient 11). The estimation bias in this case was 0.5 mmHg with an RMSE of 2.0 mmHg. These recordings indicate that our method generated nICP estimates that were within clinically acceptable accuracy compared to standard invasive methods, and that it can function in both closed-, and open-drain scenarios, in patients with different pathologies.

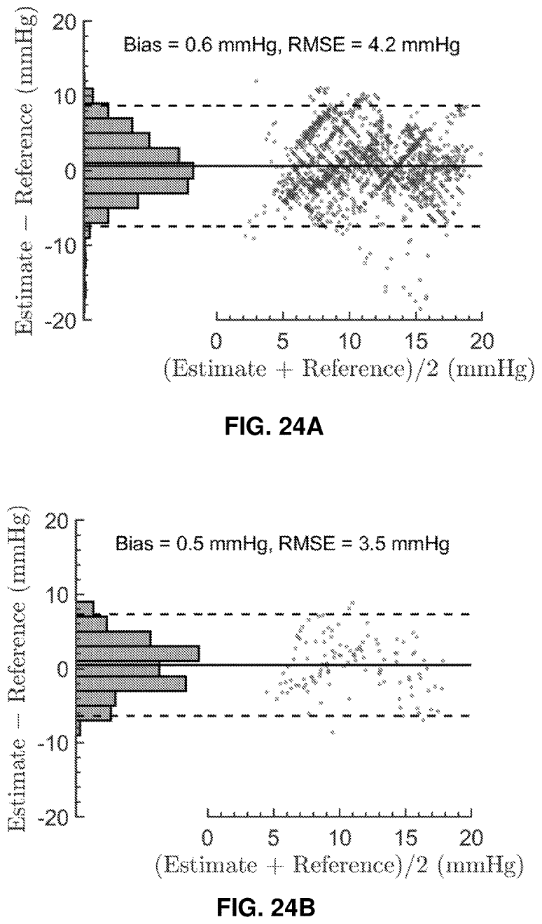

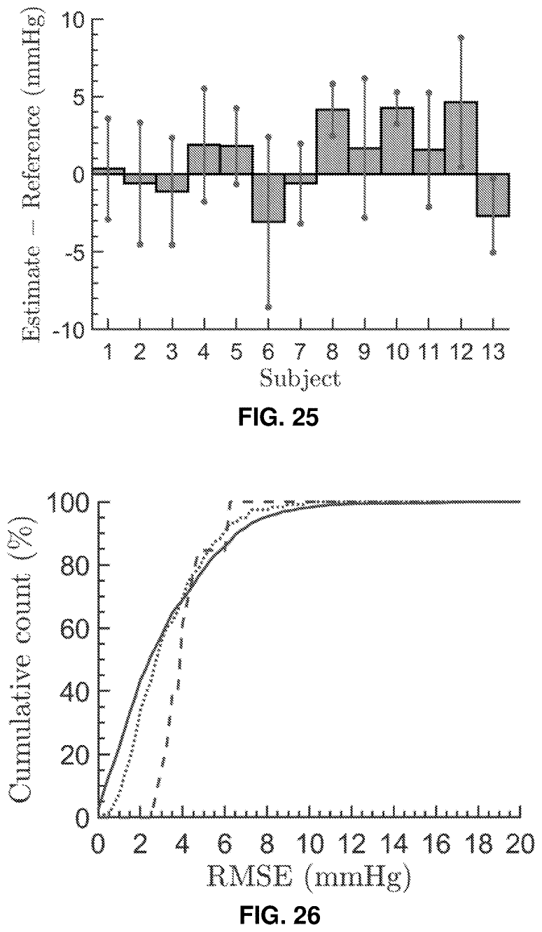

We performed a Bland-Altman analysis to quantify the overall performance of our method. The analysis was performed on a per-estimation-window and per-recording basis (FIGS. 24A and 24B). These analyses indicate that our method achieved an error of 0.6 mmHg and RMSE of 4.2 mmHg in the ensemble data with limits of agreement (bias ±1.96 standard deviation (SD)) of −7.5 and 8.7 mmHg, respectively. Likewise, the comparison on a per-recording basis revealed an estimation bias and RMSE of 0.5 and 3.5 mmHg, respectively, with limits of agreement at −6.4 and 7.3 mmHg. Finally, the per-patient estimation performance is summarized in FIG. 25. These results together indicate that our estimates are well within clinically desired tolerances.

To further gauge our method's accuracy, we computed the fraction of nICP estimates below a certain RMSE on a per-patient, per-record, and per-estimation-window basis. This analysis is illustrated in FIG. 26 and indicates that nearly 80% of all our nICP estimates were within ±5 mmHg of the invasive reference ICP measurements, indicating a strong agreement between invasive reference and noninvasive estimates.

3. Discussion

It is important to analyze the accuracy of invasive ICP measurement modalities in order to put our performance metrics of an estimation bias of 0.6 mmHg and RMSE of 4.2 mmHg in perspective. Invasive ICP monitoring modalities include clinical gold-standard external ventricular drainage (EVD) systems, and integrated (micro-transducer) sensing devices such as the Camino or Codman sensors. Performance analyses of such micro-transducers have been reported in the literature. For example, a cohort of fifteen patients, the Codman sensor had an ensemble bias of 0.3 mmHg with limits of agreement of −6.7 and 7.1 mmHg, relative to EVD measurements. That our system approaches these performance characteristics is therefore a positive indicator.

Radial arterial blood pressure measurement was the only (minimally) invasive aspect of our approach and was used simply because these measurements are readily available at the bedside. The risk of infection from arterial catheters is reported to be far less than that associated with EVDs infection rates of 5% and 10% have been previously reported in EVDs. For instances, some report infection rates of 1.5% in femoral arterial lines with 1.94 times greater risk of infection at femoral sites compared to radial sites. Also, noninvasive arterial blood pressure monitors can pave the path towards fully noninvasive ICP estimation.

Previous work first proposed the continuous-time ICP model, and also developed an associated nICP estimation routine. They reported an ensemble bias of 1.6 mmHg with an SDE of 7.6 mmHg in data from TBI patients with significant reference ICP variability. They also averaged the nICP estimates obtained from CBFV signals recorded simultaneously from left and right middle cerebral arteries, and reported that this averaging resulted in a reduced SDE of 5.9 mmHg. They were, however, unable to account for the hydrostatic pressure offset between rABP and ICP measurements as they did not have access to the height differences between the ICP and rABP pressure transducers. Also, their data were recorded solely from adult TBI patients, and thus, they did not gauge their method's performance on a wider range of pathologies.

There are several challenges in adopting model-based nICP estimation approaches in clinical practice. The rABP, for example, might not always be a faithful surrogate for the cABP owing to changes in the arterial blood pressure profile, and this may affect the nICP estimation accuracy. Likewise, the CBFV waveform cannot often be recorded from the same cerebral blood vessel, and morphological differences between CBFV signals recorded from different vessels may alter the resulting nICP estimates. Physiologically induced time delays between the rABP and CBFV signals also contribute to estimation errors. Some have investigated the time offset problem while retaining the same underlying model. In their approach, they first bandpass filter the rABP and CBFV levels to suppress their respective mean levels, before estimating nICP estimates for a range of time offsets. They analyze the resulting R and C estimates to select the final nICP estimate. On a three-patient database, the authors reported an estimation bias and SDE of −1.1 and 5.6 mmHg, respectively.

Our method differs from both these prior methods in several aspects. We accounted for height differences between the pressure transducers, while not employing any mean suppression of rABP and CBFV signals in our approach. Our method includes a strategy to encounter unknown physiologically-induced time offsets between rABP and CBFV signals. Additionally, we have introduced a simple AR model of ICP dynamics that helps in tracking ICPs over long recording durations without overly relying on the prior distribution employed in the initial stage of the method. Our method generates a probability distribution of ICP values, that can be used to determine estimation-confidence metrics, a feature not provided by the other methods. We tested our method on patients with diverse pathologies. Unlike prior methods, we did not have access to simultaneous bilateral CBFV recordings, and thus our method might achieve better performance characteristics in such scenarios.

An attractive feature of our approach is that it retains its interpretability due to the underlying physiologic model. Several possible time offsets between the rABP and CBFV are considered, which helps address the challenge posed by unknown (and patient-specific) time offsets between these signals. Estimation is performed within a Bayesian framework, which helps increase the method's resilience to structured errors that may be introduced, for instance by differences between rABP and cABP morphology, and also to unstructured errors due to signal noise and motion artifacts in recorded data. An encouraging aspect of our approach is that it achieved an RMSE of nearly 4 mmHg with parameter choices that are applicable to diverse patient populations. Specifically, our prior distribution for baseline estimation spanned negative ICPs, as well as high ICPs. Also, we set γₘ close to +1, with a correspondingly large σₘ_{ᵥ}² for generalizability to data with large ICP variability.

The nICP estimation method proposed in this paper does not require calibration to invasive ICP measurements. Our system can thus be used as a screening tool for identifying patients suffering from elevated ICP without resorting to invasive and painful procedures such as lumbar punctures. In addition to monitoring patients suffering from neurological diseases, our system can be useful in monitoring intra-operative cerebral perfusion and autoregulation. Both inadequate and excessive cerebral perfusion has been shown to be a cause of post-operative delirium. Surgical procedures such as coronary artery bypass graft (CABG) typically do not employ concurrent invasive ICP monitoring, and thus cerebral perfusion pressure cannot be directly measured. Cerebral perfusion pressure derived from our nICP estimates can be potentially used to ameliorate this problem.

Such clinical translation of our method will require implementing it for real-time operation. This is a straightforward prospect because the method employs a set of deterministic causal mathematical operations. Another possible future course of exploration can be to noninvasively estimate ICP pulsatility. While the mean ICP is clinically most relevant, ICP pulsatility has also been proposed to be an important clinical indicator. Additional work may focus on testing our proposed method on a larger dataset comprising subjects with more diverse pathologies, age and gender. We have used routinely measured rABP recordings for estimating ICPs in our clinical dataset, and future validation of the method could also involve noninvasive blood pressure monitors. Work may also focus on harnessing information in the estimated model coefficients, αₘ and βₘ, both for monitoring a subject's cerebral autoregulation status, and for assessing nICP estimation confidence on a window-by-window basis. Our model incorporates an AR description of temporal evolution of the ICP. Similar descriptions for αₘ and βₘ can be developed and integrated into our model, albeit at the cost of increasing computational complexity of the resulting ICP estimation algorithm. In the present work, we used preset values of the hyper-parameters γₘ and σₘ_{ᵥ}² in our ICP prediction model. While these preset values were shown to function well across our data, work can focus on automatically and robustly identifying these parameters from longitudinal nICP estimates.

Continuous noninvasive ICP monitoring can benefit a large number of patients. The nICP estimation framework proposed in this paper hopefully paves the way towards developing a reliable, continuous, realtime, accurate, and fully noninvasive ICP monitoring device to improve neurocritical care across the world.

4. Methods

4.1 Data Processing

All extracted data segments were first passed through a set of preprocessing steps. First, a coarse time alignment step was applied between the rABP and the CBFV signals to account for time delays introduced by different measurement devices. This time offset was obtained by computing the cross-correlation between the rABP and the CBFV signals, and the lag with the highest cross-correlation coefficient was selected as the desired offset. Doing so, however, did not account for physiologically-induced time offsets between rABP and CBFV. Following this time offset correction step, the signals were resampled to a common 125 Hz to compensate for any underlying sampling frequency discrepancies. Finally, the baseline rABP was adjusted to account for differences in ICP and rABP transducer heights. We then passed the signals through an out-of-band-noise removal stage. The rABP and CBFV trends were first extracted via a 256-tap moving-average filter. These trends were subtracted from the rABP and CBFV signals, respectively, and the resulting detrended signals were filtered by a 128-tap bandpass filter with cutoffs at 0.5 and 16 Hz. The trend removed in the first stage was then added back to the filter output to restore the original DC levels. We then assigned an in-band-noise, ag to individual non-overlapping, twenty-beat data windows. An estimation window was marked as noisy if the cross-correlation coefficient between any of the corresponding rABP and CBFV beats was below the threshold of 0.2.

4.2 FIR Model Derivation

Our FIR model of cerebral hemodynamics is a discrete-time approximation of the continuous-time model developed earlier in our group. For the m^{th} estimation window, this continuous-time model is of the form

$$q ( t ) = \frac{1}{{R}_{m}} ( {p}_{a} ( t ) - I [ m ] ) + {C}_{m} \frac{d}{d t} ( {p}_{a} ( t ) - I [ m ] )$$

where the model resistance, Rₘ, and compliance, Cₘ, are assumed to remain constant during the data window. Approximating the derivative operation by first-order finite-differences, and denoting discrete-time sampling indices with n,

$$\begin{matrix}q [ n ] = \frac{1}{{R}_{m}} ( {p}_{a} [ n ] ) - I [ m ] ) + {C}_{m} {f}_{s} \{ ( {p}_{a} [ n ] - I [ m ] ) - ( {p}_{a} [ n - 1 ] - I [ m ] ) \} \\ = ( \frac{1}{{R}_{m}} + \frac{C}{T} ) ( {p}_{a} [ n ] - I [ m ] ) - {C}_{m} {f}_{s} ( {p}_{a} [ n - 1 ] - I [ m ] ) \\ = {α}_{m} ( {p}_{a} [ n ] - I [ m ] + {β}_{m} ( {p}_{a} ( N + 1 ) - I [ m ] )\end{matrix}$$

where fₛ is the sampling rate (=125 Hz for our data),

$${α}_{m} = \frac{1}{{R}_{m}} + {C}_{m} {f}_{s} , and {β}_{m} = - {C}_{m} {f}_{s} .$$

This FIR filter, along with the ICP AR process description of Equation 2 form our complete model of cerebral hemodynamics that is employed in the proposed method. The method itself comprises two stages, that internally employ a common model-solving routine. We describe this routine next, and then proceed to describing the two stages.

4.3 Model-Based Bayesian Estimation Routine

This routine is employed in both baseline determination and subsequent ICP tracking, and it solves the model in Equation 1 for a range of candidate ICP and time offset pairs. It takes as input preprocessed rABP and CBFV signals in individual estimation windows, and computes nICP estimates by treating each window independently. Since all operations are confined to individual data windows, we omit the window index, m, for clarity in the remainder of this section.

We select the time offset range such that the CBFV peaks are constrained to lead the corresponding rABP systolic peaks whilst ensuring that the diastolic points of the two waveforms are aligned with each other (FIG. 6). This is because the underlying Windkessel-like model dictates that both signals should start rising simultaneously at the onset of systole, with the CBFV rising faster to reach its peak before the rABP. To compensate for possibly inaccurate beat detections and modeling inaccuracies, we allowed the diastolic indices of the two waveforms to differ by at most three samples (≈25 ms). All time offsets in which these two criteria are met form the time offset scan range.

To form the ICP scan range, we start scanning from an ICP of −10 mmHg, as negative ICPs are physiologically possible. We scan the ICP in increments of 1 mmHg this granularity was deemed sufficient for clinical diagnostic purposes and stop at the mean rABP in the estimation window, as the ICP cannot exceed the rABP itself.

For each ICP and time offset pair, we compute estimates for α and β in a least-squared-error sense

$${[ {\hat{α}}^{I , d} , {\hat{β}}^{I , d} ]}^{T} = {( {Φ}^{IT} {Φ}^{I} )}^{†} {Φ}^{IT} {Φ}^{d} \\ where {q}^{d} = {[ q [ 2 - d ] , … , q \{ N - d ] ]}^{T} \\ {Φ}^{I} = [ \begin{matrix}{p}_{a} [ 2 ] - I & {p}_{a} [ 1 ] - I \\ ⋮ & ⋮ \\ {p}_{a} [ N ] - I & {p}_{a} [ N - 1 ] - I\end{matrix} ] \qquad ( 3 )$$

Here, the † symbol represents a matrix pseudo-inverse, N denotes the number of samples in the estimation window, and I and d signify the solution's dependence on the candidate ICP and time offset values, respectively. The corresponding residual-error norm is given by ζ^{I,d}=|Φ^{I}[\hat{α}^{I,d},\hat{β}^{I,d}]^{T}−q^{d}|₂.

We define a likelihood distribution [symbol] (I, d)

$$ℒ ( I , d ) = \frac{1}{{S}_{ℒ}} \times exp \{ - {( \frac{{ζ}^{I , d}}{θ} )}^{2} \} \\ θ = {min}_{I , d} {ζ}^{I , d} \qquad ( 4 )$$

where [symbol] is chosen so that [symbol] (I, d)sums to one. This formulation assigns high likelihood to (I, d) pairs that result in a small residual error, and a conversely low likelihood to pairs with large residual error norms. To subsequently employ a prior distribution across the ICP, we marginalize [symbol] (I, d) across the time offsets to generate a one-dimensional likelihood distribution defined across the ICP only

$$ℒ ( I ) = {\sum }_{d} ℒ ( I , d ) \qquad ( 5 )$$

This distribution's mode, Î_{L}, and variance, σ_{L}² are computed according to

$${\hat{I}}_{L} = {argmax}_{I} ℒ ( I ) \\ {σ}_{L}^{2} = {\sum }_{I} \{ {( I - {\sum }_{I} I \times ℒ ( I ) )}^{2} \times ℒ ( I ) \} \qquad ( 6 )$$

Finally, an a posteriori distribution is generated by combining the likelihood distribution with our prior belief

$$Pr ( I \vert {p}_{a} , {q}_{v} ) = \frac{1}{{S}_{p}} \times Pr ( I ) ℒ ( I ) \qquad ( 7 )$$

where Sₚ is chosen so that the distribution sums to one. The mode and variance of this combined distribution are denoted as Î_{C}, and variance, σ_{C}², respectively.

In our method, we used a prior belief of the form

$$Pr ( I ) = \{ \begin{matrix}\frac{1}{S} \times {\sum }_{k = 1}^{2} \frac{{w}_{k}}{\sqrt{2 π {σ}_{k}}} exp \{ - \frac{1}{2} {( \frac{I - {μ}_{k}}{{σ}_{k}} )}^{2} \} , I ∈ {I}_{r a n g e} \\ 0 , I ∉ {I}_{r a n g e}\end{matrix} \qquad ( 8 )$$

w₁, w₂ ∈[0, 1], subject to the constraint w₁+w₂=1

where I_{range} denotes the ICP scan range, and S is chosen such that Pr(I) sums to unity. We selected a representative subset of 46 twenty-beat estimation windows from three subjects (1, 2, and 7) to derive parameters for this distribution, and found the mean ICP and standard deviation to be 13.6 and 2.8 mmHg, respectively. We then set μ₁=13.6 mmHg to model low ICPs, and set σ₁=10 mmHg−a value larger than the ICP standard deviation in the 46 estimation windows—to model greater variance in ICPs. Additionally, we set μ₂=50 mmHg and σ₂=20 mmHg to model high ICPs. We set w₁=0.8 and w₂=0.2 by noting that the mean ICP exceeded 30 mmHg in 20% of the data records used in previous work from our group.

4.4 Baseline ICP Estimation

To establish a baseline, we compute a posteriori mode estimates in the first M_{b}=5 data windows where the corresponding in-band-noise flag is not raised. The mode estimates are averaged to yield the baseline, I_{B}. We set M_{b} to five to ensure that a hundred beats (or more than a minute) of data are analyzed before setting the baseline. The nICP estimates, Î[m], in these estimation windows, are set equal to the corresponding a posteriori mode estimates, Î_{C}[m].

The baseline ICP is passed to the subsequent tracking stage. This stage uses mode estimates of the likelihood distribution. This amounts to using a uniform prior belief, and is done to reduce dependence on the initial prior distribution. Using a uniform belief, however, also increases the chances of error-prone nICP estimates. We therefore developed a tracking framework that filters the changes in nICP estimates computed with the uniform prior belief. This filtering is achieved by combining observed nICP estimates with model-predicted changes obtained with our AR process model.

The baseline computation stage passes its baseline ICP estimate to the tracking stage. A reference nICP and variance obtained solely from the likelihood distribution are also computed according to

I_{L,ref}=Î_{L}[m_{TS}]

σ_{L,ref}²=σ_{L}²[m_{TS}]

where m_{TS} is the last selected estimation window's index. These values are used to initialize the tracking filter which is described next.

4.5 Tracking Changes in the ICP

Filtered ICP-change estimates are computed by combining observed and model-predicted changes in ICP for m≥m_{TS}. In the following description, we denote the observed nICP changes as ΔO[m+1]. Their estimated variances are denoted as σ_{ΔO}² [m+1]. We denote the model-predicted ICP changes as ΔP[m+1] and their estimated variances as σ_{ΔP}² [m+1]. Likewise, the filtered ICP-change estimates are denoted as [symbol] [m+1] and their variance estimates as [symbol] [m+1].

Assuming that likelihood distributions of successive estimation windows are statistically independent, the observed nICP change (with the uniform prior) and its variance are

ΔO[m+1]=Î_{L}[m+1]−Î_{L}[m]

σ_{ΔO}²[m+1]=σ_{L}²[m+1]−σ_{L}²[m] (9)

where Î_{L} [m_{TS}] and σ_{L}²[m_{TS}] are initialized to I_{L,ref} and σ_{L,ref}², respectively. The variance estimates are upper bounds on the true variances because, by virtue of the independence assumption, the covariance terms have not been included. We compensated for this by using relatively large values of σᵥ². Also, in estimation windows where the in-band-noise flag is raised, Î_{L} [m] is set to Î_{L}[m−1], and σ_{L}² [m] is set to ϵ, where ϵ=10⁻⁹ to arrest any drifts induced by a series of noisy estimation windows. Next, we compute the model-predicted ICP change and its variance as

ΔP[m+1]=γₘ [symbol] [m]

σ_{ΔP}²[m+1]=γₘ² [symbol] [m]−σᵥ² (10)

where the prediction is made using the filtered change estimate, [symbol] [m], of the previous window. To initialize this computation at m=m_{TS}, we set [symbol] [m_{TS}] and [symbol] [m_{TS}] to 0 mmHg.

Once both model-predicted and observed ICP changes and their variances have been computed, they are combined such that

$$χ = \frac{{σ}_{Δ P}^{2} [ m + 1 ]}{{σ}_{Δ P}^{2} [ m + 1 ] + {σ}_{Δ O}^{2} [ m + 1 ]} \\ {σ}_{Δ O}^{2} [ m + 1 ] = x {σ}_{Δ O}^{2} [ m + 1 ] \\ \hat{Δ I} [ m + 1 ] = ( 1 - x ) Δ P [ m + 1 ] + x Δ O [ m + 1 ] \qquad ( 11 )$$

The resulting filtered change, [symbol] [m+1], is added to [symbol] [m] to yield the final nICP estimate,

Î[m+1]=Î[m]+ [symbol] [m+1] (12)

where I_{B} is used instead of Î[m_{TS}] in the first iteration.

In this formulation, Equation 11 can be seen to merge the predicted and observed estimates of the inter-estimation-window ICP change by assigning greater weight to the estimate with lesser variance. This Kalman-filter like process is repeated for subsequent estimation windows to yield nICP estimates with greatly reduced dependence on initial prior information.

The terms “program” or “software” are used herein in a generic sense to refer to any type of computer code or set of processor-executable instructions that can be employed to program a computer or other processor to implement various aspects of embodiments as discussed above. Additionally, it should be appreciated that according to one aspect, one or more computer programs that when executed perform methods of the disclosure provided herein need not reside on a single computer or processor, but may be distributed in a modular fashion among different computers or processors to implement various aspects of the disclosure provided herein.

Processor-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in one or more non-transitory computer-readable storage media in any suitable form. For simplicity of illustration, data structures may be shown to have fields that are related through location in the data structure. Such relationships may likewise be achieved by assigning storage for the fields with locations in a non-transitory computer-readable medium that convey relationship between the fields. However, any suitable mechanism may be used to establish relationships among information in fields of a data structure, including through the use of pointers, tags or other mechanisms that establish relationships among data elements.

Also, various inventive concepts may be embodied as one or more processes, of which examples have been provided. The acts performed as part of each process may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, and/or ordinary meanings of the defined terms.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having,” “containing”, “involving”, and variations thereof, is meant to encompass the items listed thereafter and additional items.

Having described several embodiments of the techniques described herein in detail, various modifications, and improvements will readily occur to those skilled in the art. Such modifications and improvements are intended to be within the spirit and scope of the disclosure. Accordingly, the foregoing description is by way of example only, and is not intended as limiting. The techniques are limited only as defined by the following claims and the equivalents thereto.

1. A system comprising:
at least one hardware processor; and
at least one non-transitory computer-readable storage medium storing processor-executable instructions that, when executed by the at least one hardware processor, cause the at least one hardware processor to perform:
obtaining a first set of data identifying arterial blood pressure and cerebral blood flow velocity of a patient during a first period of time;
estimating an initial intracranial pressure value for the patient by using a statistical model to compute a posterior distribution of intracranial pressure values based on the first set of data and a prior distribution of intracranial pressure values;
obtaining a second set of data identifying arterial blood pressure and cerebral blood flow velocity of the patient during a second period of time;
estimating an updated intracranial pressure value for the patient by determining a change in intracranial pressure of the patient based on the second set of data and the initial intracranial pressure value; and
outputting information indicating the updated intracranial pressure value.

2. The system of claim 1, wherein the statistical model includes at least one of a parameter representing cerebrovascular resistance, a parameter representing cerebrovascular compliance, a parameter representing intracranial pressure, and a parameter representing inertance.

3. The system of claim 1, wherein the statistical model relates arterial blood pressure and cerebral blood flow velocity to intracranial pressure.

4. The system of claim 1, wherein determining the change in intracranial pressure of the patient is performed at least in part by using the statistical model and the second set of data to estimate at least one value for a set of parameters of the statistical model.

5. The system of claim 1, wherein the at least one hardware processor is further configured to perform:
predict a change in intracranial pressure for at least a third period of time after the second period of time based on the at least one value for the set of parameters of the statistical model.

6. The system of claim 4, wherein determining the change in intracranial pressure of the patient is performed at least in part by estimating the at least one value for the set of parameters of the statistical model using the statistical model and the second set of data to evaluate a plurality of intracranial pressure values at a plurality of time offsets corresponding to a time shift between arterial blood pressure measurements and cerebral blood flow velocity measurements obtained from the patient.

7. The system of claim 1, wherein obtaining the first set of data includes obtaining arterial blood pressure and cerebral blood flow velocity of the patient over a plurality of cardiac cycles.

8. The at least one non-transitory computer-readable storage medium of claim 17, wherein the at least one hardware processor is further configured to perform:
estimating a series of intracranial pressure values for the patient by estimating changes in intracranial pressure of the patient based on the second set of data and combining the estimated changes in intracranial pressure with the estimated initial intracranial pressure value.

9. The at least one non-transitory computer-readable storage medium of claim 8, wherein estimating the series of intracranial pressure values further comprises dynamically updating an estimated intracranial pressure value during the second period of time as the second set of data is obtained.

10. The system of claim 1, wherein estimating the initial intracranial pressure value further comprises using the statistical model to compute a posterior distribution of intracranial pressure values based on a likelihood of intracranial pressure given the first set of data and the prior distribution of intracranial pressure values.

11. The system of claim 1, wherein the at least one hardware processor is further configured to perform:
predicting a set of values for a physiological signal for the patient using the statistical model and data obtained from the patient, the predicting performed at least in part by using the statistical model and the data to evaluate a plurality of intracranial pressure values at a plurality of time offsets between arterial blood pressure and cerebral blood flow velocity;
generating a set of prediction errors by comparing the predicted set of values for the physiological signal to a portion of the data corresponding to the physiological signal; and
computing the likelihood of intracranial pressure based on the set of prediction errors.

12. The system of claim 11, wherein computing the likelihood of intracranial pressure further comprises determining, for each of the plurality of time offsets, a likelihood of intracranial pressure distribution for the time offset from a subset of prediction errors associated with using the time offset in computing the physiological signal.

13. The system of claim 12, wherein computing the likelihood of intracranial pressure further comprises combining the likelihood of intracranial pressure distribution for each of the plurality of time offsets to determine the likelihood of intracranial pressure.

14. The method of claim 18, wherein estimating the initial intracranial pressure value further comprises:
computing at least one intracranial pressure value using the first set of data at a time interval within the first period of time;
determining a metric indicative of the level of noise in the first set of data during the time interval; and
selecting, based on comparing the metric to a threshold value, to include the at least one intracranial pressure value in estimating the intracranial pressure value.

15. The method of claim 18, wherein estimating an updated intracranial pressure value further comprises:
computing, for a time interval within the second duration of time, a predicted change in intracranial pressure for the patient based on a subset of the second set of data corresponding to at least one time interval preceding the time interval;
computing, for the time interval, a data-derived change in intracranial pressure for the patient based on a subset of the second set of data corresponding to the time interval;
determining an estimated change in intracranial pressure based on the predicted change in intracranial pressure and the data-derived change in intracranial pressure; and
using the estimated change in intracranial pressure to estimate the updated intracranial pressure value.

16. The method of claim 18, wherein the prior distribution of intracranial pressure values corresponds to data obtained from at least one person other than the patient.

17. At least one non-transitory computer-readable storage medium storing processor-executable instructions that, when executed by at least one hardware processor, cause the at least one hardware processor to perform:
obtaining a first set of data identifying arterial blood pressure and cerebral blood flow velocity of a patient during a first period of time;
estimating an initial intracranial pressure value for the patient by using a statistical model to compute a posterior distribution of intracranial pressure values based on the first set of data and a prior distribution of intracranial pressure values;
obtaining a second set of data identifying arterial blood pressure and cerebral blood flow velocity of the patient during a second period of time;
estimating an updated intracranial pressure value for the patient by determining a change in intracranial pressure of the patient based on the second set of data and the initial intracranial pressure value; and
outputting information indicating the updated intracranial pressure value.

18. A method, comprising:
using at least one hardware processor to perform:
obtaining a first set of data identifying arterial blood pressure and cerebral blood flow velocity of a patient during a first period of time;
estimating an initial intracranial pressure value for the patient by using a statistical model to compute a posterior distribution of intracranial pressure values based on the first set of data and a prior distribution of intracranial pressure values;
obtaining a second set of data identifying arterial blood pressure and cerebral blood flow velocity of the patient during a second period of time;
estimating an updated intracranial pressure value for the patient by determining a change in intracranial pressure of the patient based on the second set of data and the initial intracranial pressure value; and
outputting information indicating the updated intracranial pressure value.

19. A system comprising:
at least one hardware processor; and
at least one non-transitory computer-readable storage medium storing processor-executable instructions that, when executed by the at least one hardware processor, cause the at least one hardware processor to perform:
obtaining data that includes an arterial blood pressure waveform and a cerebral blood flow velocity waveform of a patient during a first period of time, wherein the arterial blood pressure waveform and the cerebral blood flow velocity waveform are obtained at different locations of the patient;
estimating an intracranial pressure value for the patient by using a statistical model to compute a posterior distribution of intracranial pressure values based on a likelihood of intracranial pressure given the data and a prior distribution of intracranial pressure values, wherein using the statistical model includes using at least one time offset value between the arterial blood pressure waveform and the cerebral blood flow velocity waveform; and
outputting information indicating the updated intracranial pressure value.

20. The system of claim 19, wherein using the statistical model to compute the posterior distribution further comprises aligning in time the arterial blood pressure waveform and the cerebral blood flow velocity waveform.

21. The system of claim 20, wherein aligning the arterial blood pressure waveform and the cerebral blood flow velocity waveform further comprises constraining the alignment, for at least one cardiac cycle, such that a systolic peak in cerebral blood flow velocity occurs prior to a systolic peak in arterial blood pressure.

22. The system of claim 21, wherein aligning the arterial blood pressure waveform and the cerebral blood flow velocity waveform further comprises constraining the alignment, for at least one cardiac cycle, such that a diastolic point in cerebral blood flow velocity occurs at substantially the same time as a diastolic point in arterial blood pressure.

23. The system of claim 20, wherein the at least one hardware processor is further configured to perform:
selecting the at least one time offset value from a plurality of time offset values based on the alignment of the arterial blood pressure waveform and the cerebral blood flow velocity waveform meeting a set of physiological constraints.

24. The system of claim 23, wherein the set of physiological constraints include that a systolic peak in cerebral blood flow velocity occurs prior to a systolic peak in arterial blood pressure.

25. The system of claim 23, wherein the set of physiological constraints include that a diastolic point in cerebral blood flow velocity occurs at substantially the same time as a diastolic point in arterial blood pressure.

26. The system of claim 19, wherein the statistical model includes at least one of a parameter representing cerebrovascular resistance, a parameter representing cerebrovascular compliance, a parameter representing intracranial pressure, and a parameter representing inertance.

27. The system of claim 19, wherein the statistical model relates arterial blood pressure and cerebral blood flow velocity to intracranial pressure.

28. The system of claim 19, wherein obtaining the data includes obtaining arterial blood pressure and cerebral blood flow velocity of the patient over a plurality of cardiac cycles.

29. The at least one non-transitory computer-readable storage medium of claim 37, wherein the at least one hardware processor is further configured to perform:
estimating a series of intracranial pressure values for the patient by estimating changes in intracranial pressure of the patient using the statistical model and the data.

30. The at least one non-transitory computer-readable storage medium of claim 29, wherein estimating the series of intracranial pressure values further comprises dynamically updating an estimated intracranial pressure value as data is obtained during a second period of time.

31. The system of claim 19, wherein estimating the initial intracranial pressure value further comprises using the statistical model to compute a posterior distribution of intracranial pressure values based on a likelihood of intracranial pressure given the data and the prior distribution of intracranial pressure values.

32. The system of claim 31, wherein the at least one hardware processor is further configured to perform:
predicting a set of values for a physiological signal for the patient using the statistical model and the at least one time offset value, the predicting performed at least in part by evaluating a plurality of intracranial pressure values at each of the at least one time offset value;
generating a set of prediction errors by comparing the predicted set of values for the physiological signal to a portion of the data corresponding to the physiological signal; and
computing the likelihood of intracranial pressure based on the set of prediction errors.

33. The system of claim 32, wherein computing the likelihood of intracranial pressure further comprises determining, for each of the at least one time offset value, a likelihood of intracranial pressure distribution for the time offset from a subset of prediction errors associated with using the time offset value in computing the physiological signal.

34. The system of claim 33, wherein computing the likelihood of intracranial pressure further comprises combining the likelihood of intracranial pressure distribution for each of the at least one time offset in determining the likelihood of intracranial pressure.

35. The method of claim 38, wherein estimating the intracranial pressure value further comprises:
computing at least one intracranial pressure value using the data at a time interval within the first period of time;
determining a metric indicative of the level of noise in the data during the time interval; and
selecting, based on comparing the metric to a threshold value, to include the at least one intracranial pressure value in estimating the intracranial pressure value.

36. The method of claim 38, wherein the prior distribution of intracranial pressure values corresponds to data obtained from at least one person other than the patient.

37. At least one non-transitory computer-readable storage medium storing processor-executable instructions that, when executed by at least one hardware processor, cause the at least one hardware processor to perform:
obtaining data that includes an arterial blood pressure waveform and a cerebral blood flow velocity waveform of a patient during a first period of time, wherein the arterial blood pressure waveform and the cerebral blood flow velocity waveform are obtained at different locations of the patient;
estimating an intracranial pressure value for the patient by using a statistical model to compute a posterior distribution of intracranial pressure values based on a likelihood of intracranial pressure given the data and a prior distribution of intracranial pressure values, wherein using the statistical model includes using at least one time offset value between the arterial blood pressure waveform and the cerebral blood flow velocity waveform; and
outputting information indicating the updated intracranial pressure value.

38. A method, comprising:
using at least one hardware processor to perform:
obtaining data that includes an arterial blood pressure waveform and a cerebral blood flow velocity waveform of a patient during a first period of time, wherein the arterial blood pressure waveform and the cerebral blood flow velocity waveform are obtained at different locations of the patient;
estimating an intracranial pressure value for the patient by using a statistical model to compute a posterior distribution of intracranial pressure values based on a likelihood of intracranial pressure given the data and a prior distribution of intracranial pressure values, wherein using the statistical model includes using at least one time offset value between the arterial blood pressure waveform and the cerebral blood flow velocity waveform; and
outputting information indicating the updated intracranial pressure value.
